SEQUENCE LISTING (73 sequences)

SEQ ID NO: 1 - DNA, 72 bp, Homo sapiens
  source 1..72: mol_type = genomic DNA
tgccgtgactcggattcgaaccgaggttgctgcggccacaacgcagagtactaaccactatacgatcacggc

SEQ ID NO: 2 - DNA, 74 bp, Homo sapiens
  source 1..74: mol_type = genomic DNA
cgtccctgggtgggctcgaaccaccaacctttcggttaacagccgaacgcgctaaccgattgcgccacagagac

SEQ ID NO: 3 - DNA, 75 bp, Homo sapiens
  source 1..75: mol_type = genomic DNA
gccgtgatcgtatagtggttagtactctgcgttgtggccgcagcaacctcggttcgaatccgagtcacggcacca

SEQ ID NO: 4 - DNA, 77 bp, Homo sapiens
  source 1..77: mol_type = genomic DNA
gtctctgtggcgcaatcggttagcgcgttcggctgttaaccgaaaggttggtggttcgagcccacccagggacgcca

SEQ ID NO: 5 - DNA, 76 bp, Homo sapiens
  source 1..76: mol_type = genomic DNA
ccttcgatagctcagctggtagagcggaggactgtagatccttaggtcgctggttcgattccggctcgaaggacca

SEQ ID NO: 6 - DNA, 75 bp, Homo sapiens
  source 1..75: mol_type = genomic DNA
tcctcgttagtatagtggtgagtatccccgcctgtcacgcgggagaccggggttcgattccccgacggggagcca

SEQ ID NO: 7 - DNA, 72 bp, Homo sapiens
  source 1..72: mol_type = genomic DNA
gtaaatatagtttaaccaaaacatcagattgtgaatctgacaacagaggcttacgaccccttatttacccca

SEQ ID NO: 8 - DNA, 76 bp, Homo sapiens
  source 1..76: mol_type = genomic DNA
tagattgaagccagttgattagggtgcttagctgttaactaagtgtttgtgggtttaagtcccattggtctagcca

SEQ ID NO: 9 - DNA, 69 bp, Homo sapiens
  source 1..69: mol_type = genomic DNA
ggtaaaatggctgagtgaagcattggactgtaaatctaaagacaggggttaggcctctttttaccacca

SEQ ID NO: 10 - DNA, 71 bp, Homo sapiens
  source 1..71: mol_type = genomic DNA
aaggtattagaaaaaccatttcataactttgtcaaagttaaattataggctaaatcctatatatcttacca

SEQ ID NO: 11 - DNA, 77 bp, Homo sapiens; identical to SEQ ID NO: 4
  source 1..77: mol_type = genomic DNA

SEQ ID NO: 12 - DNA, 77 bp, Homo sapiens
  source 1..77: mol_type = genomic DNA
gtctctgtggcgcaatcggttagcgcattcggctgttaaccgaaaggttggtggttcgagcccacccagggacgcca

SEQ ID NO: 13 - DNA, 77 bp, Homo sapiens
  source 1..77: mol_type = genomic DNA
gtctctgtggcgcaatcggttagcgcgttcggctgttaaccgaaaggttggtggttcgatcccacccagggacgcca

SEQ ID NO: 14 - DNA, 77 bp, Homo sapiens
  source 1..77: mol_type = genomic DNA
gtctctgtggcgcaatcggttagcgcgttcggctgttaactgaaaggttggtggttcgagcccacccagggacgcca

SEQ ID NO: 15 - DNA, 77 bp, Homo sapiens
  source 1..77: mol_type = genomic DNA
gtctctgtggcgcaatgggttagcgcgttcggctgttaaccgaaaggttggtggttcgagcccatccagggacgcca

SEQ ID NO: 16 - DNA, 76 bp, Homo sapiens
  source 1..76: mol_type = genomic DNA
ggctccgtggcgcaatggatagcgcattggacttctaattcaaaggttccgggttcgagtcccggcggagtcgcca

SEQ ID NO: 17 - DNA, 76 bp, Homo sapiens
  source 1..76: mol_type = genomic DNA
ggctctgtggcgcaatggatagcgcattggacttctaattcaaaggttgtgggttcgagtcccaccagagtcgcca

SEQ ID NO: 18 - DNA, 76 bp, Homo sapiens
  source 1..76: mol_type = genomic DNA
ggctctgtggcgcaatggatagcgcattggacttctaattcaaaggttgtgggttcgaatcccaccagagtcgcca

SEQ ID NO: 19 - DNA, 75 bp, Homo sapiens
  source 1..75: mol_type = genomic DNA
ggccccatggtgtaatggttagcactctggactttgaatccagcgatccgagttcaaatctcggtgggacctcca

SEQ ID NO: 20 - DNA, 75 bp, Homo sapiens
  source 1..75: mol_type = genomic DNA
ggtcccatggtgtaatggttagcactctggactttgaatccagcgatccgagttcaaatctcggtgggacctcca

SEQ ID NO: 21 - DNA, 75 bp, Homo sapiens
  source 1..75: mol_type = genomic DNA
ggtcccatggtgtaatggttagcactctggactttgaatccagcaatccgagttcgaatctcggtgggacctcca

SEQ ID NO: 22 - DNA, 75 bp, Homo sapiens
  source 1..75: mol_type = genomic DNA
tcccatatggtctagcggttaggattcctggttttcacccaggtggcccgggttcgactcccggtatgggaacca

SEQ ID NO: 23 - DNA, 75 bp, Homo sapiens
  source 1..75: mol_type = genomic DNA
tcccacatggtctagcggttaggattcctggttttcacccaggcggcccgggttcgactcccggtgtgggaacca

SEQ ID NO: 24 - DNA, 75 bp, Homo sapiens
  source 1..75: mol_type = genomic DNA
tccctggtggtctagtggctaggattcggcgctttcaccgccgcggcccgggttcgattcccggccagggaacca

SEQ ID NO: 25 - DNA, 75 bp, Homo sapiens
  source 1..75: mol_type = genomic DNA
tccctggtggtctagtggctaggattcggcgctttcaccgccgcggcccgggttcgattcccggtcagggaacca

SEQ ID NO: 26 - DNA, 11 bp, Roseburia sp.
  source 1..11: mol_type = genomic DNA
cggctgttaac

SEQ ID NO: 27 - DNA, 11 bp, Roseburia sp.
  source 1..11: mol_type = genomic DNA
gccctgtcacg

SEQ ID NO: 28 - DNA, 11 bp, Roseburia sp.
  source 1..11: mol_type = genomic DNA
agattgtggct

SEQ ID NO: 29 - DNA, 11 bp, Roseburia sp.
  source 1..11: mol_type = genomic DNA
agactgtaaat

SEQ ID NO: 30 - DNA, 11 bp, Bacteroides dorei
  source 1..11: mol_type = genomic DNA
caagattttca

SEQ ID NO: 31 - DNA, 11 bp, Bifidobacterium longum
  source 1..11: mol_type = genomic DNA
ccagactttca

SEQ ID NO: 32 - DNA, 11 bp, Carnobacterium maltaromaticum
  source 1..11: mol_type = genomic DNA
ccgccctttca

SEQ ID NO: 33 - DNA, 11 bp, Clostridium beijerinckii
  source 1..11: mol_type = genomic DNA
ccaccctttca

SEQ ID NO: 34 - DNA, 11 bp, Faecalibacterium prausnitzii
  source 1..11: mol_type = genomic DNA
cggccctttca

SEQ ID NO: 35 - DNA, 11 bp, Lachnoclostridium phytofermentans
  source 1..11: mol_type = genomic DNA
ccgccctttca

SEQ ID NO: 36 - DNA, 11 bp, Roseburia intestinalis
  source 1..11: mol_type = genomic DNA
tcgccctttca

SEQ ID NO: 37 - DNA, 11 bp, Bacteroides dorei
  source 1..11: mol_type = genomic DNA
caggttttggt

SEQ ID NO: 38 - DNA, 11 bp, Bifidobacterium longum
  source 1..11: mol_type = genomic DNA
gggtctttgga

SEQ ID NO: 39 - DNA, 11 bp, Carnobacterium maltaromaticum
  source 1..11: mol_type = genomic DNA
cagactttgac

SEQ ID NO: 40 - DNA, 11 bp, Clostridium beijerinckii
  source 1..11: mol_type = genomic DNA
tggactttgac

SEQ ID NO: 41 - DNA, 11 bp, Faecalibacterium prausnitzii
  source 1..11: mol_type = genomic DNA
gggactttgac

SEQ ID NO: 42 - DNA, 11 bp, Lachnoclostridium phytofermentans
  source 1..11: mol_type = genomic DNA
aggactttgac

SEQ ID NO: 43 - DNA, 11 bp, Roseburia intestinalis
  source 1..11: mol_type = genomic DNA
tggattttgat

SEQ ID NO: 44 - DNA, 11 bp, Bacteroides dorei
  source 1..11: mol_type = genomic DNA
agctgactacg

SEQ ID NO: 45 - DNA, 11 bp, Bifidobacterium longum
  source 1..11: mol_type = genomic DNA
agctgactacg

SEQ ID NO: 46 - DNA, 11 bp, Carnobacterium maltaromaticum
  source 1..11: mol_type = genomic DNA
acttgactacg

SEQ ID NO: 47 - DNA, 11 bp, Clostridium beijerinckii
  source 1..11: mol_type = genomic DNA
taccgctacgg

SEQ ID NO: 48 - DNA, 11 bp, Faecalibacterium prausnitzii
  source 1..11: mol_type = genomic DNA
gactgactacg

SEQ ID NO: 49 - DNA, 11 bp, Lachnoclostridium phytofermentans
  source 1..11: mol_type = genomic DNA
gtctggctacg

SEQ ID NO: 50 - DNA, 11 bp, Roseburia intestinalis
  source 1..11: mol_type = genomic DNA
acttggctacg

SEQ ID NO: 51 - DNA, 11 bp, Bacteroides dorei
  source 1..11: mol_type = genomic DNA
ccggattccgg

SEQ ID NO: 52 - DNA, 11 bp, Bifidobacterium longum
  source 1..11: mol_type = genomic DNA
tctgtctccgg

SEQ ID NO: 53 - DNA, 11 bp, Carnobacterium maltaromaticum
  source 1..11: mol_type = genomic DNA
taagattccgg

SEQ ID NO: 54 - DNA, 11 bp, Faecalibacterium prausnitzii
  source 1..11: mol_type = genomic DNA
gttcggctccg

SEQ ID NO: 55 - DNA, 11 bp, Roseburia intestinalis
  source 1..11: mol_type = genomic DNA
ccggactccga

SEQ ID NO: 56 - DNA, 11 bp, Bacteroides dorei
  source 1..11: mol_type = genomic DNA
agaatttccta

SEQ ID NO: 57 - DNA, 11 bp, Bifidobacterium longum
  source 1..11: mol_type = genomic DNA
ccgctctccta

SEQ ID NO: 58 - DNA, 11 bp, Clostridium beijerinckii
  source 1..11: mol_type = genomic DNA
atcccctccta

SEQ ID NO: 59 - DNA, 11 bp, Faecalibacterium prausnitzii
  source 1..11: mol_type = genomic DNA
acacgcctcct

SEQ ID NO: 60 - DNA, 11 bp, Roseburia intestinalis
  source 1..11: mol_type = genomic DNA
actcgcctcct

SEQ ID NO: 61 - DNA, 11 bp, Bacteroides dorei
  source 1..11: mol_type = genomic DNA
aacgcccttct

SEQ ID NO: 62 - DNA, 11 bp, Bifidobacterium longum
  source 1..11: mol_type = genomic DNA
atgtgacttct

SEQ ID NO: 63 - DNA, 11 bp, Carnobacterium maltaromaticum
  source 1..11: mol_type = genomic DNA
atccgccttct

SEQ ID NO: 64 - DNA, 11 bp, Clostridium beijerinckii
  source 1..11: mol_type = genomic DNA
aacgcccttct

SEQ ID NO: 65 - DNA, 11 bp, Faecalibacterium prausnitzii
  source 1..11: mol_type = genomic DNA
aactgccttct

SEQ ID NO: 66 - DNA, 11 bp, Lachnoclostridium phytofermentans
  source 1..11: mol_type = genomic DNA
atcggccttct

SEQ ID NO: 67 - DNA, 11 bp, Roseburia intestinalis
  source 1..11: mol_type = genomic DNA
aacggccttct

SEQ ID NO: 68 - DNA, 11 bp, Bacteroides dorei
  source 1..11: mol_type = genomic DNA
ctacgttcggg

SEQ ID NO: 69 - DNA, 11 bp, Bifidobacterium longum
  source 1..11: mol_type = genomic DNA
gcttcgttcgg

SEQ ID NO: 70 - DNA, 11 bp, Faecalibacterium prausnitzii
  source 1..11: mol_type = genomic DNA
cctcgttcggg

SEQ ID NO: 71 - DNA, 11 bp, Lachnoclostridium phytofermentans
  source 1..11: mol_type = genomic DNA
gctacgttcgg

SEQ ID NO: 72 - DNA, 11 bp, Roseburia intestinalis
  source 1..11: mol_type = genomic DNA
gctgcgttcgg

SEQ ID NO: 73 - DNA, 11 bp, Bacteroides dorei
  source 1..11: mol_type = genomic DNA
aagtttcctaa

(19) United States
(12) Patent Application Publication
(10) Pub. No.: US 2025/0283146 A1
(43) Pub. Date: Sep. 11, 2025

(54) ANALYSIS OF RNA MODIFICATIONS

(71) Applicant: The University of Chicago, Chicago, IL (US)

(72) Inventors: Tao Pan, Chicago, IL (US); Christopher Katanski, Chicago, IL (US); Christopher Watkins, Chicago, IL (US); Samuel Miller, Chicago, IL (US)

(73) Assignee: The University of Chicago, Chicago, IL (US)

(21) Appl. No.: 18/862,467

(22) PCT Filed: May 4, 2023
(86) PCT No.: PCT/US2023/066607

§ 371 Date: Nov. 1, 2024

Related U.S. Application Data
(60) Provisional application No. 63/338,160, filed on May 4, 2022.

(51) Int. Cl. C12Q 1/6806; C12Q 1/6809; C12Q 1/6869

(52) U.S. Cl. CPC C12Q 1/6806; C12Q 1/6809; C12Q 1/6869; C12Q 2600/156; C12Q 2600/166

(57) ABSTRACT

In aspects, the invention provides a method of detecting a nucleotide modification in a sample comprising RNA, the method comprising: reacting RNA in the sample with periodate to form periodate-treated RNA in the sample, sequencing periodate-treated RNA in the sample, and detecting a modification signature in the sequence.

### CROSS-REFERENCE TO RELATED APPLICATION

This application claims the priority benefit of U.S. Provisional Patent Application No. 63/338,160, filed May 4, 2022, the disclosure of which is incorporated herein by reference in its entirety.

### STATEMENT REGARDING FEDERALLY SPONSORED RESEARCH OR DEVELOPMENT

This invention was made with Government support under Grant Number NIH RM1HG008935 awarded by the National Institutes of Health and Grant Number BC191198 awarded by the Congressionally Directed Medical Research Programs of the Department of Defense. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 66,294 Byte Extensible Markup Language (xml) file named “767182_SequenceListing.xml,” created on May 3, 2023, with a final production date of May 4, 2023.

### BACKGROUND

Queuosine (Q) is a 7-deaza-7-aminomethyl-cyclopentenediol derivative present at the wobble anticodon position (34 in tRNA nomenclature) of tRNAs of Tyr, His, Asn, and Asp. Uniquely among the ˜50 modifications in the human RNA, queuosine tRNA modification is synthesized de novo in bacteria, whereas in mammals the substrate for Q-modification in tRNA is queuine, the catabolic product of the Q-base of gut bacteria. Q34 is known to enhance decoding speed, tune decoding accuracy in translation, and modulate tRNA fragment biogenesis.

Currently, the methods of detecting and quantifying Q-modification in tRNA include radioactive guanine exchange, liquid chromatography-mass spectrometry (LC/MS), acryloylaminophenyl boronic acid (APB) or acid denaturing gel electrophoresis. There is a need for new methods of detecting Q and other modifications in tRNA.

### BRIEF SUMMARY

In aspects, the disclosure provides a method of detecting a nucleotide modification in a sample comprising RNA, the method comprising: reacting RNA in the sample with periodate to form periodate-treated RNA in the sample, sequencing periodate-treated RNA in the sample, and detecting a modification signature in the sequence. Additional aspects are as described herein.

### BRIEF DESCRIPTION OF THE DRAWINGS

FIG. 1 depicts Q-modification in tRNAs, and shows a gel and sequencer plots demonstrating that Q-modification generates deletion signatures after periodate treatment. FIG. 1A depicts the chemical structure of the Q-base and its proposed periodate oxidized form. FIG. 1B depicts a Northern blot of an APB gel showing the Q-modification levels in tRNA^{Asn} and tRNA^{His} samples from cells cultured in 0Q and 100Q media. “Q” indicates tRNA with, and “G” indicates tRNA without, queuosine modification. The shift in gel migration distance seen for Q-modified tRNAs is caused by the reaction of the Q-base with the boronic acid derivative used in APB gels. FIG. 1C depicts a graph of the deletion fraction seen in cDNA sequences of tRNAHis from 0Q and 100Q samples, ±periodate treatment. FIG. 1D depicts an expanded view of the graphs shown in FIG. 1C in the region ±5 nt (nucleotides) of the Q34 residue (vertical dashed line). Biological replicates are overlaid in each graph. Only data for the most abundant tRNA^{His} isodecoder is shown. N1-methylguanosine (m1G) at position 37 is another known modification that produces a deletion signature seen in the graphs. FIG. 1E depicts graphs showing mutation, insertion, and stop fraction signatures detected in the region ±5 nt to the Q34 residue, using the same samples shown in panel FIG. 1C.

FIG. 2 depicts graphs showing periodate treatment-dependent deletion signatures in cDNAs made from tRNAs. The graphs have an expanded scale relative to those shown in FIG. 1C, and display regions ±5 nt to the Q34 residue (dashed line) in each tRNA. Biological replicates are overlaid in each graph. For nuclear-encoded tRNAs, only data for the most abundant isodecoder for Asn/Tyr/Asp is shown. All residue numbers are according to the standard tRNA nomenclature, i.e., the wobble anticodon nucleotide assigned position 34. The tRNAs whose Q-modification deletion signatures are shown are: FIG. 2A, nuclear-encoded tRNA^{Asn}, FIG. 2B, mitochondrial-encoded tRNA^{His}, FIG. 2C, mitochondrial-encoded tRNA^{Asn}, and FIG. 2D, mitochondrial-encoded tRNA^{Tyr}. In addition to Q-modification deletion signatures, several tRNAs also display a deletion signature at the known modification ms²i⁶A at position 37. The ms²i⁶A deletion signature is shown in FIG. 2E for mitochondrial-encoded tRNA^{Asp}, and FIG. 2F for nuclear-encoded tRNA^{Tyr}. Another known modification that also produces a deletion signature is m1G at position 37, shown in FIG. 2G for nuclear-encoded tRNA^{Asp}. The periodate-independent deletion signature at position 37 is of unknown origin.

FIG. 3. provides additional information about the Q-modified tRNAs used in the experiments whose results are shown in FIGS. 1 and 2. In the figure, gaps were introduced in the sequences to enable alignment at wobble position 34 and to maximize sequence conservation. FIG. 3A shows the sequences of Q-modified nuclear-encoded and mitochondrial-encoded tRNAs, respectively, as their cDNA sequences. The anticodon nucleotides are shown in bold, and the region ±5 nt of position 34 is underlined. FIG. 3B shows the sequences of the 5 tRNA^{Asn} isodecoders with the highest expression levels in HEK293T cells. The anticodon nucleotides are in bold, and the ±5 nt region is underlined in the most-expressed isodecoder. Sequence differences among the isodecoders are shown as the single nucleotides in bold. FIG. 3C is a plot showing the abundance of the 5 tRNA^{Asn}isodecoders with the highest expression levels in HEK293T cells. The numbers 11-15 correspond to the tRNA sequences in FIG. 3B. Each symbol corresponds to one biological replicate sequenced separately.

FIG. 4 depicts graphs showing quantitative assessment of detected Q-modification levels in nuclear-encoded tRNA^{His}and tRNA^{Asn}. Shown are regions ±3 nt to the Q34 residue (dashed line) in each tRNA. FIG. 4A shows an overlay of the detected deletion fraction for tRNA^{His} found for 11 calibration samples that were pre-mixed with decreasing proportions of OQ and increasing proportions of 100Q RNAs, prepared as described in the Methods of Example 1.FIG. 4B shows an overlay of the detected deletion fraction for tRNA^{Asn} found for the 11 calibration samples, prepared as described for FIG. 4A. FIG. 4C shows that the deletion fraction at Q34 can be fit to the equation log2y=a+bx, where a is the intercept and b is the slope. The tRNA^{His} curve has a fit of a=−7.0, b=0.023, and r2=0.964. The tRNA^{Asn} curve has a fit of a=−6.0, b=0.025, and r2=0.985. FIG. 4D shows the Q-modification levels found in the 5 tRNA Asn isodecoders expressed at the highest levels. Each symbol corresponds to one biological replicate sequenced separately.

FIG. 5 depicts graphs and plots showing analysis of periodate treatment-dependent 2-thio tRNA modifications in biological samples without Q modification (denoted 0Q). All residue numbers are according to the standard tRNA nomenclature, i.e., the wobble anticodon nucleotide is at position 34. FIG. 5A shows the chemical structures of the 2-thio-modifications found in the tRNAs and their proposed periodate oxidized forms. Shown in FIGS. 5B-D are mutation and deletion signatures seen in regions ±5 nt from the relevant residue (dashed line) in each human tRNA. The samples are 0Q and results are shown with (solid lines) and without (dotted lines) periodate treatment. Biological replicates are overlaid in each graph. FIG. 5B shows the mutation and deletion fractions seen for the indicated mitochondrial-encoded tRNAs known to contain 5-taurinomethy-2-thio-U (τm⁵s²U) at the wobble anticodon position. FIG. 5C shows the mutation and deletion fractions seen for the indicated mitochondrial-encoded tRNAs known to contain 5-taurinomethy-U (τm⁵U) at the wobble anticodon position. FIG. 5D shows the mutation and deletion fractions seen for the indicated nuclear-encoded tRNAs known to contain 5-methoxycarbonylmethyl-2-thio-U (mcm⁵s²U) 34. Another known modification that also produces signatures is N3-methylcytosine (m3C) found at position 32 for tRNA^{Arg}(TCT). FIG. 5E shows the mcm⁵s²U34 mutation rates and abundance for isodecoders of tRNA^{Arg}(TTC), tRNA^{Gln}(TTG), and tRNA^{Glu}(TTC), with and without periodate treatment. Each symbol corresponds to one biological replicate sequenced separately.

FIG. 6 depicts graphs and plots showing analysis of periodate treatment-dependent 2-thio tRNA modifications in biological samples with Q modification (denoted 100 Q). All residue numbers are according to the standard tRNA nomenclature, i.e., the wobble anticodon nucleotide is at position 34. The samples contain the same 2-thio modifications depicted in FIG. 5A. Shown in FIGS. 6A-B are mutation and deletion signatures seen in regions ±5 nt from the relevant residue (dashed line) in each human tRNA. The samples are 100 Q, with (solid lines) and without (dotted lines) periodate treatment. Biological replicates are overlaid in each graph. FIG. 6A shows the mutation and deletion fractions seen for the indicated mitochondrial-encoded tRNAs known to contain 5-taurinomethy-2-thio-U (τm⁵s²U) at the wobble anticodon position. FIG. 6B shows the mutation and deletion fractions seen for the indicated nuclear-encoded tRNAs known to contain 5-methoxycarbonylmethyl-2-thio-U (mcm⁵s²U) 34. FIG. 6C shows the mcm⁵s²U34 mutation rates and abundance for isodecoders of tRNA^{Arg}(TCT), tRNA^{Gln}(TTG), and tRNA^{Glu}(TTC), with and without periodate treatment. Each symbol corresponds to one biological replicate sequenced separately.

FIG. 7 depicts the sequences of mcm⁵s²U34-modified human tRNA isodecoders as DNA sequences. The anticodon nucleotides are in bold, and the ±5 nt region is underlined in the isodecoder expressed at the highest level for each tRNA. Sequence differences among the isodecoders are shown as the scattered nucleotides in bold.

FIG. 8 depicts graphs and plots showing 2-thio tRNA modifications in E. coli tRNA, and response thereof to stress. Shown are mutation and deletion signatures in regions ±5 nt from the relevant residue (dashed line) in each tRNA. FIG. 8A shows the chemical structures of the 2-thio-modifications found in the tRNAs and their proposed periodate oxidized forms. FIG. 8B shows the mutation and deletion fractions found for the indicated tRNAs known to contain 5-carboxymethylaminomethyl-2-thio-U (cmnm⁵s²U) 34. FIG. 8C shows the mutation and deletion fractions found for the indicated tRNAs known to contain 2-thio-C (s²C.) at position 32. Another known modification that also produces sequence signatures is I34 in tRNA^{Arg}(ACG). FIG. 8D shows the mutation and deletion fractions found for tRNA^{His} known to contain 4-thio-U (s⁴U) at position 8. FIG. 8E shows the response of E. coli tRNA containing the s²C32 modification to exposure to stressors 2,2′-dipyridyl (DIP), hydrogen peroxide (H₂O₂), and methyl α-D-glucopyranoside (αMG). Biological replicates are shown in each plot. ***: p<10−3, ns: not significant. The X-axis shows the type of stressors (“none” corresponds to no stressor), y-axis shows the difference in mutation signature of each E. coli tRNA containing s²C32 modification.

FIG. 9. E. coli stress response of tRNA^{Gln}(TTG) and tRNA^{Glu}(TTC) containing cmnm⁵s²U34 to exposure to stressors 2,2′-dipyridyl (DIP), hydrogen peroxide (H₂O₂), and methyl α-D-glucopyranoside (αMG). The X-axis shows the type of stressors (“none” corresponds to no stressor), y-axis shows the difference in mutation signature of all E. coli tRNAs containing cmnm⁵s²U34 modification.

FIG. 10 shows the abundance of microbial 5S rRNAs from different bacterial taxa at the class level. Libraries were constructed for the same human stool sample under four treatment conditions: #is minus periodate, minus demethylase; square is plus periodate, minus demethylase; circle is minus periodate, plus demethylase; and * is plus periodate, plus demethylase.

FIG. 11 depicts the deletion fraction of tRNA Q-modifications found in reference sequences from the species indicated in FIG. 11A. FIG. 11A shows the deletion fraction at nucleotide position 34 (wobble position in tRNA anticodon) for 4 bacterial classes. Sequence reads were performed under the treatments: #is minus periodate, square is plus periodate. FIG. 11B shows the deletion fraction in the +/−5 nucleotide region surrounding position 34 of Q-modifiable tRNAs in the genus Roseburia. Sequencing was performed with (solid line) and without (dotted line) periodate treatment.

FIG. 12 depicts s²U modifications around tRNA position U34 found in reference sequences from the bacterial species indicated. Sequencing was performed with (solid line) and without (dotted line) periodate treatment.

FIG. 13 depicts s²C32 modifications around tRNA position C32 found in reference sequences from the bacterial species indicated. In the specific microbes studied, some tRNAs of the corresponding anticodons are absent so there are no sequences for them. For example, there is no tRNA^{Arg}(CCG) in C. beijerinckii, or L. phylofermentans. Sequencing was performed with (solid line) and without (dotted line) periodate treatment.

FIG. 14 depicts depicts s²C34 modifications around tRNA position C34 found in reference sequences from the bacterial species indicated. In the specific microbes studied, some tRNAs of the corresponding anticodons are absent, so there are no sequences for them. For example, there is no tRNA^{Pro}(CGG) in C. maltaromaticum or C. beijerinckii. Sequencing was performed with (solid line) and without (dotted line) periodate treatment.

### DETAILED DESCRIPTION

In aspects, the disclosure provides a method of detecting a nucleotide modification in a sample comprising RNA, the method comprising: reacting RNA in the sample with periodate to form periodate-treated RNA in the sample, sequencing periodate-treated RNA in the sample, and detecting a modification signature in the sequence. In aspects, the method further comprising performing a control sequencing reaction on a portion of RNA from the sample, wherein the control sequencing reaction is performed on RNA not treated with periodate. In aspects, the nucleotide modification is (a) substitution with queuosine (Q-modification) or (b) substitution of an oxygen atom at the 2-position of a pyrimidine nucleotide with a sulfur atom (2-thio modification).

In aspects, the nucleotide modification is detected as the presence of a mutation signature or a deletion signature in the sequence. As used herein, a “signature” refers to a distinctive base misincorporation (mutation) caused in nucleic acid sequencing by the base modification itself, or by its derivative that results from various chemical or enzymatic treatment. A signature can be a deletion, insertion, or stop in the RNA-seq data. The signature results when the reading mechanism of the reverse transcriptase enzyme encounters the unique chemical structure(s) of modification(s) in the template RNA.

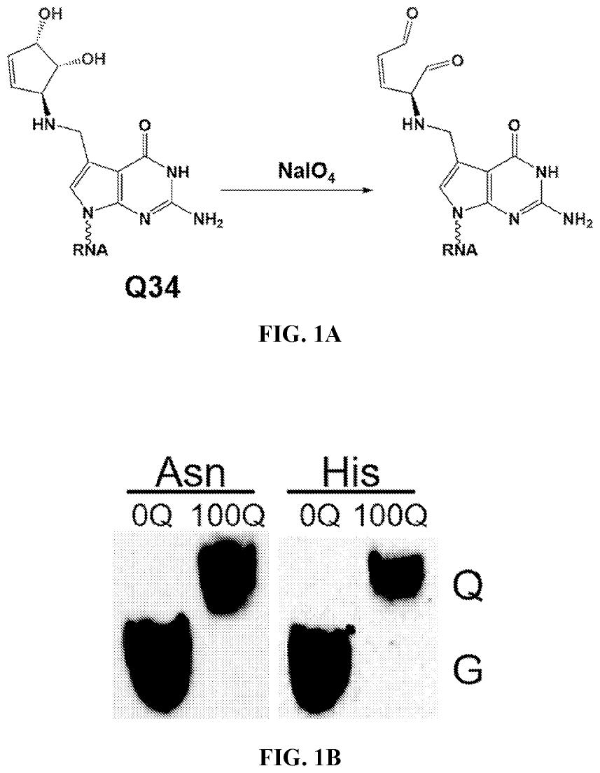

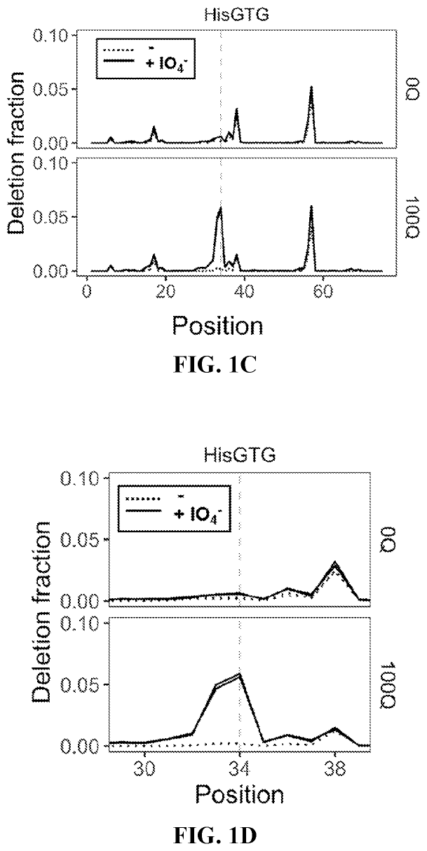

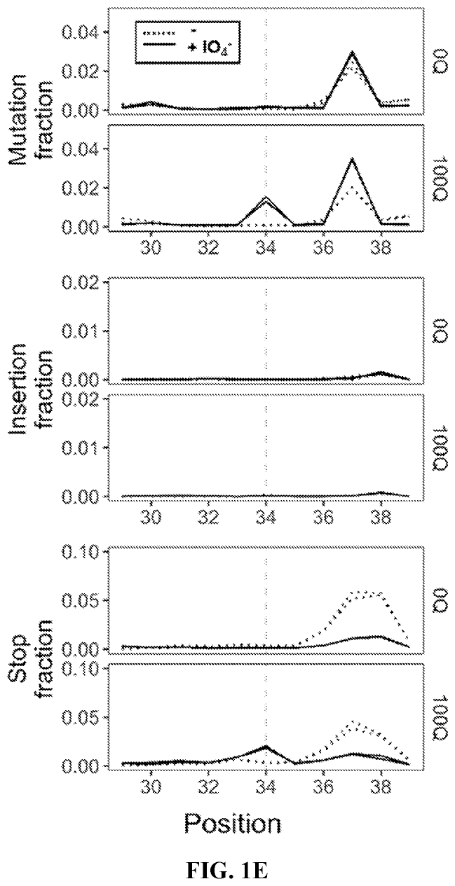

In aspects, the nucleotide modification is Q-modification. Queuosine (Q) is a 7-deaza-7-aminomethyl-cyclopentenediol derivative present at the wobble anticodon position (34 in tRNA nomenclature) of the tRNAs of Tyr, His, Asn, and Asp amino acids (see FIG. 1A, Q34). Queuosine tRNA modification is synthesized de novo in bacteria, whereas in mammals the substrate for Q-modification in tRNA is queuine, the catabolic product of the Q-base of gut bacteria. The G34 guanine base is replaced with queuine in the four tRNAs by a two-component enzyme encoded in the mammalian genome to produce Q-modified tRNA. Q34 is known to enhance decoding speed, tune decoding accuracy in translation, and modulate tRNA fragment biogenesis.

In mammals, gut availability of queuine affects virulence of resident gut microbes and modulates cancer growth. Q-modification levels in tRNA are especially high in human brain tissues, and queuine plays a role in resistance to cancer metabolism and neuronal damage. Since queuine must be scavenged from the gut and Q-tRNA modification is directly involved in protein biosynthesis, queuine and Q-tRNA modification present a clear connection between the gut microbiome and host proteostasis.

Current methods of detecting and quantifying Q-modification in tRNA have limitations. Guanine exchange is only useful to quantify the total Q levels in all tRNAs. LC/MS can precisely analyze Q-modification in individual tRNAs, but it requires large amounts of input material and it is difficult to quantify Q-fraction. Gel electrophoresis methods can quantify Q-modification in individual tRNAs, but still require micrograms of total RNA and are done for only one tRNA species at a time. Additionally, neither LC/MS nor gels have sufficient resolution for studies of tRNA isodecoders in mammals. The term “isodecoders” refers to tRNA sequences that share the same anticodon but differ in the sequence of the body of the tRNA.

In aspects, the presence of a deletion signature in the sequence at the site of the Q-modification is detected. In aspects, the periodate-treated RNA is sequenced using high throughput DNA sequencing.

High throughput (also known as Nextgen or deep) DNA sequencing has become a versatile tool to study many RNA modifications. A variety of DNA sequencing instruments and platforms are commercially available. A preferred system for performing DNA sequencing is the NGS (Next Generation Sequencing) System of Illumina, Inc. In sequencing RNA using DNA sequencing, the RNA is used as a template for the enzyme reverse transcriptase, which makes a cDNA copy of the template RNA. Any suitable reverse transcriptase (RT) can be used, for example, TGI RT, AMV RT, ThermoScript™M RT (InvitrogenTM), MMLV RT, SuperScript™M IV RT (Invitrogen™M) and the like. In aspects, the reverse transcriptase can be SuperScript™ IV RT (Invitrogen™M).

A modification signature is found in the DNA copy of the RNA template that is produced by reverse transcriptase. The RNA sequences can be reported as either the sequence of the DNA copy thereof, i.e., a sequence comprising the DNA bases A, G, C and T, or as the RNA sequence itself, i.e., with a sequence comprising the RNA bases A, G, C and U. Q-modification itself has not been found to leave a detectable signature in standard RNA-seq procedures, despite the large chemical moiety attached to the 7-position of the Q-base (see FIG. 1A, Q34). Thus, Q-modification is detected in the DNA copy of the periodate-treated RNA, and not in the DNA copy of the control (untreated) RNA.

Periodate is known to oxidize cis-diol groups into aldehydes. Periodate oxidation can be used to study tRNA aminoacylation levels by chromatography, microarrays, or sequencing. The Q-base has a cis-diol group that is a known substrate for periodate oxidation (see reaction depicted in FIG. 1A), a common reaction used to confirm the presence of Q-modification in APB gel electrophoresis. In a tRNA-seq procedure to measure tRNA charging levels, periodate treatment is often a step in the sequencing library construction before reverse transcription (Evans, M. E., Clark, W. C., Zheng, G. and Pan, T. (2017) Determination of tRNA aminoacylation levels by high-throughput sequencing. Nucleic Acids Res, 45, e133; Behrens, A., Rodschinka, G. and Nedialkova, D. D. (2021) High-resolution quantitative profiling of tRNA abundance and modification status in eukaryotes by mim-tRNAseq. Mol Cell, 81, 1802-1815). These studies, however, could not be used for the detection of Q-modification. In the Evans et al. paper, no control of RNA minus-periodate from the same samples was sequenced, and the Q-modification levels of these samples were unknown. In the Behrens et al. paper, only Saccharomyces cerevisiae samples were periodate treated, and S. cerevisiae naturally do not have Q-modification in their tRNAs.

The discovery that oxidized Q-base induces a deletion signature in the reverse transcriptase (RT) reaction in the sequencing library construction was unexpected. Without being bound by any particular hypothesis, it is thought that Q-base affects anticodon-codon pairing through altering anticodon loop geometry and increasing its rigidity. In vitro studies of codon-anticodon complexes show a 3-fold increase in stabilization of Q-U pairings over G-U, while pairings with C were destabilized. The 5-member ring of the Q-base is located in the major groove of the RNA-DNA hybrid in the active site of reverse transcriptase. Periodate oxidation opens the ring which may lead to increased flexibility and steric occupancy of the oxidized moiety in the major groove, thereby inducing the RT to skip the oxidized Q nucleotide.

In aspects, the method of detecting a nucleotide modification in RNA further comprises quantifying the fraction of RNA having Q-modification. In aspects, quantifying the fraction of RNA comprises comparing a detected Q-level in the RNA to a calibration curve established from RNA with no Q-modification (0Q) and with full Q-modification (100Q).

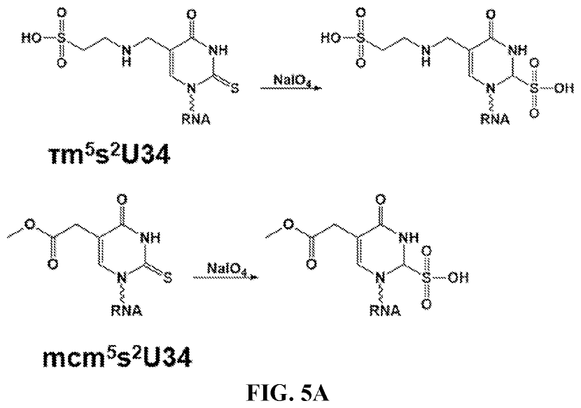

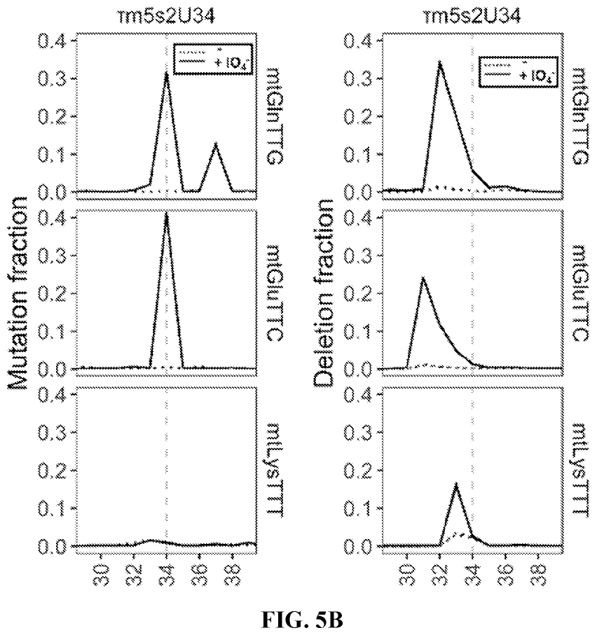

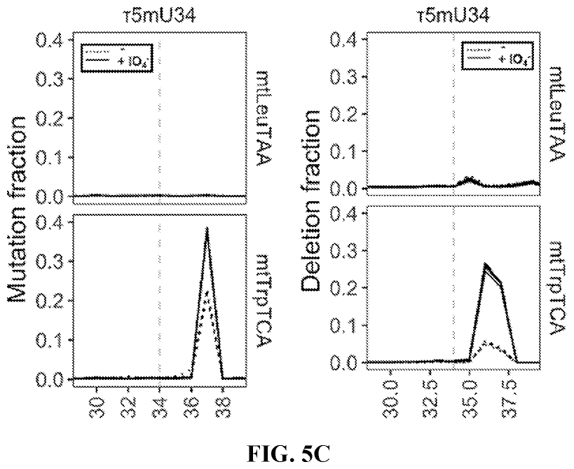

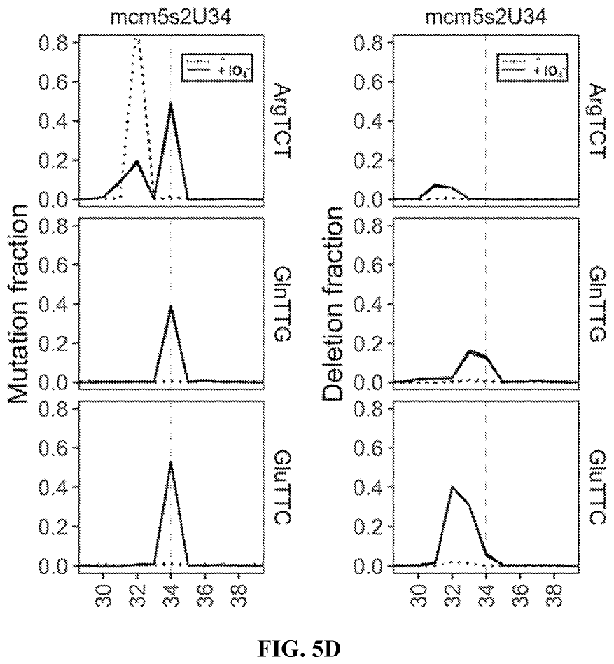

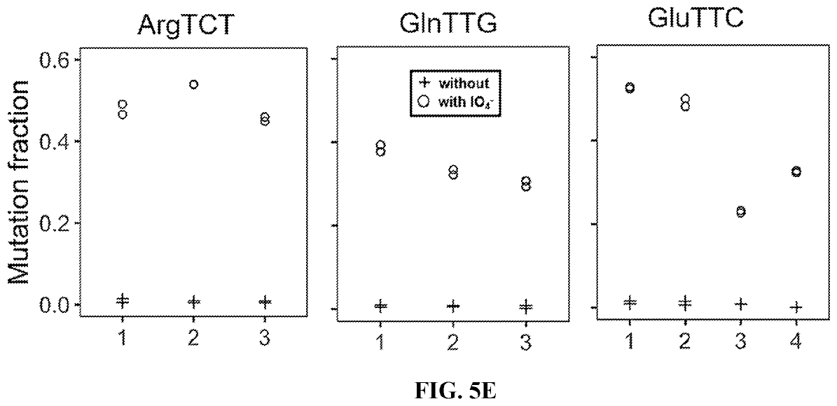

In aspects, the nucleotide modification is 2-thio modification. Thio-modifications are widespread in mammalian and bacterial tRNAs. In human tRNAs, the direct substitutions of the oxygen atom with sulfur at either the 2-(2-thio, s²) or 4-(4-thio, s⁴) position of pyrimidines occurs in the wobble anticodon uridine (U34) of several nuclear-encoded tRNAs and several mitochondrial-encoded tRNAs. Examples of 2-thio modifications are shown as Tm⁵s²U34 and mcm⁵s²U34in FIG. 5A.

In E. coli tRNAs, the s² modification occurs in the wobble anticodon uridines of several tRNAs, as well as in cytosine at position 32 (s²C32) in the anticodon loop of several other tRNAs. Furthermore, many E. coli tRNAs also contain the s⁴U modification at position 8, between the acceptor and D stem regions. The anticodon s²U34 modifications are always accompanied by additional modifications at C5, and the thio-modification plays a role in the decoding efficiency of the C-versus U-ending codons of Gln, Glu, Lys tRNAs, and the AGY codon of Arg tRNA. The s²C32 modification plays a role in selective decoding of Arg codons. The s⁴U8 modification is a UV sensor that initiates a UV response in E. coli.

Periodate is known to oxidize sulfides and thiol groups (reactions shown in FIG. 5A), which could alter the sequencing signatures of thio modifications present in some in tRNAs. The 2-thio modification is located in the minor groove of the DNA-RNA hybrid formed in the active site of the reverse transcriptase enzyme. 2-thio oxidation may alter the proof-reading mechanism of the reverse transcriptase, resulting in detectable signatures in cDNA sequencing. In aspects, 2-thio modifications are detected by the presence of (i) a mutation signature in the RNA sequence at the site of the 2-thio modification and/or (ii) a deletion signature in the RNA sequence near the site of the 2-thio modification (see FIG. 5).

The RNA molecule used in the method may be any suitable RNA molecule. In aspects, the RNA is from a mammal or a bacterium. The mammal may be a human. The bacterium may be any suitable bacterium, e.g., E. coli. In aspects, the RNA is total RNA, e.g., the complete complement of RNA molecules isolated from cells. In aspects, the RNA is tRNA. In aspects, the tRNA is nuclear-encoded tRNA, i.e., cytosolic tRNA, or mitochondrial-encoded tRNA.

The following includes certain aspects of the disclosure.
1. A method of detecting a nucleotide modification in a sample comprising RNA, the method comprising: reacting RNA in the sample with periodate to form periodate-treated RNA in the sample, sequencing periodate-treated RNA in the sample, and detecting a modification signature in the sequence.
2. The method of aspect 1, further comprising performing a control sequencing reaction on a portion of RNA from the sample, wherein the control sequencing reaction is performed on RNA not treated with periodate.
3. The method of aspect 1 or aspect 2, wherein the modification signature detected is the presence of a mutation signature or a deletion signature in the sequence.
4. The method of any one of aspects 1-3, wherein the nucleotide modification is (a) substitution with queuosine (Q-modification) or (b) substitution of an oxygen atom at the 2-position of a pyrimidine nucleotide with a sulfur atom (2-thio modification).
5. The method of any one of aspects 1-4, wherein the nucleotide modification is Q-modification.
6. The method of aspect 5, wherein the presence of a deletion signature in the sequence at the site of Q-modification is detected.
7. The method of any one of aspects 1-6, further comprising quantifying the fraction of RNA having Q-modification.
8. The method of aspect 7, wherein quantifying the fraction of RNA comprises comparing a detected Q-level in the RNA to a calibration curve established from RNA with no Q-modification (0Q) and with full Q-modification (100Q).
9. The method of any one of aspects 1-4, wherein the nucleotide modification is 2-thio modification.
10. The method of aspect 9, wherein the presence of (i) a mutation signature in the sequence at the site of the 2-thio modification and/or (ii) a deletion signature in the sequence near the site of the 2-thio modification is detected.
11. The method of any one of aspects 1-10 wherein the RNA is from a mammal or a bacterium.
12. The method of any one of aspects 1-11 wherein the RNA is total RNA, tRNA, nuclear-encoded tRNA or mitochondrial-encoded tRNA.
13. The method of any one of aspects 1-12, wherein the periodate-treated RNA is sequenced using high throughput DNA sequencing.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following examples further illustrate the disclosure but, of course, should not be construed as in any way limiting its scope.

### EXAMPLE 1

### Methods

The following methods were used in analysis of queuosine and 2-thio RNA modifications, in accordance with aspects of the disclosure.

### HEK293T Cell Growth

HEK293T cells were cultured with complete DMEM medium under normal conditions. OQ HEK293T cells were obtained by culturing the cells with dialyzed FBS for certain passages, and 100Q HEK293T cells were obtained by treating 0Q cells with 1 μM queuine for 24 hours (8). Briefly, HEK293T cells were grown in complete DMEM medium (Cytiva Hyclone SH30022.01) with 10% dialyzed FBS (Thermo Fisher Scientific 26400044) and 1% Penicillin-Streptomycin (Thermo Fisher Scientific 15070063) to 80% confluency and passaged. TRIzol reagent (Thermo Fisher Scientific 15596026) was used to extract total RNAs at each passage by following the manufacturer's manual. Q levels in tRNA^{His/Asn} were constantly examined at each passage by APB gel-based Northern blot. Q modification fractions of tRNA^{His/Asn} dropped to below detection after ˜10 passages; these cells were designated as 0Q. 100Q cells were obtained by culturing 0Q cells to 60%-80% confluency, followed by incubation with 1 μM queuine for 24 hours.

### Northern Blot of APB Gels

Northern blots were performed as previously described (Zhang, W., Xu, R., Matuszek, Z., Cai, Z. and Pan, T. (2020) Detection and quantification of glycosylated queuosine modified tRNAs by acid denaturing and APB gels. RNA, 26, 1291-1298). Three μg of total RNA were added to each microcentrifuge tube and diluted to 9 μL with H2O. 1 μL of 1 M Tris-HC1 (pH 9.0) was added to the tube with mixing, followed by incubation at 37° C. for 30 min to deacylate tRNAs. 10 μL 2×RNA loading dye (8 M Urea, 0.1 M EDTA, 0.05% Bromophenol blue, 0.05% Xylene cyanol) were added to each tube. All samples were loaded to a pre-run, hand-cast 10% denaturing PAGE gel containing 0.5% (g/ml) acrylamidophenylboronic acid (APB). The gel was run in the 4° C. cold room using 1×TAE buffer at 18 W for ˜2-3 h until the xylene cyanol band was ˜1-2 cm from the bottom. The gel area containing the target tRNAs was saved and a Hybond-XL membrane (GE Healthcare, RPN303S) slightly larger than the gel was put on top of the gel and the gel remove from the plate with caution. Dry RNA transfer was then performed using a gel dryer (Bio-Rad, 1651745) for 4 h at 80° C. The gel and membrane were separated by soaking in distilled water. The RNA was crosslinked to the membrane by UV exposure for two times at 254 nm, each time 1200 mJ. The membrane was then blocked for 2×30 minutes with hybridization buffer (20 mM phosphate, pH 7, 300 mM NaCl, 1% SDS) at room temperature. The membrane was incubated with 50 mL 3 pmol/mL biotinylated tRNA probes for 16 h at 60° C. in the UVP Hybridizer Oven (Analytik Jena 95-0030-01), followed by washing with 50 mL washing buffer (20 mM phosphate, pH 7, 300 mM NaCl, 2 mM EDTA, and 0.1% SDS) for 2×30 min in the UVP Hybridizer Oven. The membrane was then incubated with streptavidin-HRP conjugate (Genscript M00091) in 30 mL hybridization buffer (1:5,000-1:10,000 dilution) for 30 min at room temperature, followed by three washes for 5 min each in 25 mL washing buffer. The membrane was then transferred to plastic wrap with the RNA-side facing up. Peroxidase-detection reagents 1 and 2 (Bio-Rad 1705061) were mixed (0.1 mL per 1 cm² membrane) and applied to the top of the membrane by pipetting. The membrane was incubated with the reagent mixture for 5 min in the dark. The membrane was then transferred to a new piece of plastic wrap to remove extra detection reagent. The membrane was scanned using the ChemiDoc imaging system (Bio-Rad) and the data was analyzed using ImageLab (Bio-Rad).

The oligonucleotide probe sequences were: tRNA^{His}. 5′-biotin-TGCCGTGACTCGGATTCGAACCGAGGTTGCTGCGGCCACAACGCAGAGTACTAACC ACTATACGATCACGGC [SEQ ID NO: 1]; tRNA^{Asn.}. 5′-biotin-CGTCCCTGGGTGGGCTCGAACCACCAACCTTTCGGTTAACAGCCGAACGCGCTAACC GATTGCGCCACAGAGAC [SEQ ID NO: 2].

E. coli Growth and RNA Extraction:

E. coli MG1655 cells were grown in LB to an A600 of 0.4 before subjecting the culture to stress conditions. Cells were harvested by centrifuging 25 mL culture for 1 min at 12,000 RCF and decanting the media. Mock-treated cells, 25 mL, were left to grow for 10 min. Iron depletion stress was done by adding 2,2′-dipyridl (DIP) to 25 mL cells to a 250 μM final concentration, for 10 min. Hydrogen peroxide stress was done by adding H₂O₂ to 25 mL cells to a final concentration of 0.5%, for 10 min. Glucose phosphate stress was done by adding α-methyl glucoside-6-phosphate (αMG) to 25 mL cells to a final concentration of 1 mM, for 10min. Cells were harvested by centrifugation at 3,000×g for 5 min, and resuspended in 0.5 mL ice cold lysis buffer (150 mM KCI, 2 mM EDTA, 20 mM HEPES pH 7.5), then flash frozen in liquid nitrogen. RNA was extracted by a hot acid-phenol protocol. Briefly, 0.5 mL of acid-buffer phenol (pH 4.5 citrate) was added to frozen samples. Samples were incubated in a heat block at 50° C. with shaking for 30 min. The aqueous phase was then removed and subjected to another round of phenol extraction, followed by 2 rounds of chloroform extraction, and finally precipitated with 2 μl of 15 mg/ml glycoblue (ThermoFisher AM9515), 300 mM sodium acetate, and 3 volumes of ethanol. Samples were incubated for 1 hour at −80° C., then centrifuged at maximum speed (20 k RCF) for 45 min to pellet RNA. Pellets were washed twice with 70% ethanol, then resuspended in water.

### Periodate Treatment

### One-Pot Deacylation and β-Elimination for tRNA Charging

For RNA that was periodate treated, up to 500 ng of total RNA in 7 μL was used for optional one-pot beta-elimination prior to library construction. To begin, 1 μL of 90 mM sodium acetate buffer, pH 4.8 was added to 7 μL input RNA. Next, 1 μL of freshly prepared 150 mM sodium periodate solution was added for a reaction condition of 16 mM NaIO4, 10 mM NaOAc pH 4.8. Periodate oxidation proceeded for 30 min at room temperature. Oxidation was quenched with addition of 1 μL of 0.6 M ribose to 60 mM final concentration and incubated for 5 minutes. Next, 5 L of freshly prepared 100 mM sodium tetraborate, pH 9.5 was added for a final concentration of 33 mM. This mixture was incubated for 30 min at 45° C. To stop β-elimination and perform 3′-end repair, 5 L of T4 PNK mix (200 mM TrisHCI pH 6.8, 40 mM MgCl2, 4 U/μL T4 PNK, from New England Biolabs) was added to the reaction, and incubated at 37° C. for 20 min. T4 PNK was heat inactivated by incubating at 65° C. for 10 min. The 20 μL reaction mixture can be used directly in the first bar-code ligation by adding 30 μL of a ligation master mix, as described below.

### Standard tRNA Deacylation

For RNA that was not periodate-treated, total RNA was prepared for library construction by deacylation in a solution of 100 mM Tris-HCl, pH 9.0 at 37° C. for 30 minutes, followed by neutralization with addition of sodium acetate, pH 4.8 to a final concentration of 180 mM. Deacylated RNA was then ethanol-precipitated and resuspended in water, or desalted using a Oligo Clean-and-Concentrator™M spin column from Zymo Research.

### Barcode Ligation and Multiplexing

Samples were incubated overnight at 16° C. in a 50 μL ligation solution with the following final concentrations: 15% PEG 8000, 1×NEB T4 RNA ligase I buffer, 50 μM ATP, 5% DMSO, 1 mM hexammine cobalt (III) chloride, 0.8 μM barcode ligation oligo, and 1 U/μL NEB T4 RNA ligase I.

After overnight ligation, 50 μL of 100 mM EDTA were added to each sample to inactivate the ligase. The samples were then combined and 8 μL of Streptavidin MyOne Cl Dynabeads from ThermoFisher per sample were added to the combined samples. The biotinylated samples were then allowed to bind to the beads for 15 minutes. After binding, the beads were magnetized according to the instructions for Invitrogen Streptavidin coated MyOne™M Cl dynabeads (https://www. thermofisher.com/order/catalog/product/65001). The supernatant was removed, and the samples were washed once with a high salt Tween wash buffer (1 M NaCl, 0.1% Tween 20, and 20 mM TrisHCI, pH 7.4) and once with low salt wash buffer (100 mM NaCl, 20 mM Tris-HCl, pH 7.4).

The RT reaction was done with Superscript IV at 55° C. for 10 min, then further incubated at 37° C. overnight. After ligation of another DNA oligonucleotide that contains the primer binding site for Illumina index primers, PCR was performed using standard Illumina index primers. The PCR products were sequenced using Illumina NovaSEQ.

### Calibration Samples

The queuosine calibration samples were mixed using a varying amounts of a combination of 0% queuosine (queuosine-depleted) HEK total RNA and 100% queuosine (queuosine-abundant) HEK total RNA to a final volume of 10 μL. (Queuosine modification levels were quantified by Northern blot.) The calibration samples ranged from 0% queuosine to 100% queuosine, in 10% intervals.

Bead-bound samples produced after the barcode ligation and multiplexing steps, as described above, were resuspended in 40 μL of deionized, autoclaved water and then 10 μL of a solution of 0.25 M NaIO4, 0.5 M NaOAc/HOAc, pH 5 (final concentration: 50 mM NaIO4,0.1 M NaOAc/HOAc, pH 5) were added. The reaction proceeded at room temperature for 30minutes and was quenched by addition of 10 μL of 1 M ribose for 5 minutes. After quenching, the samples were washed as described previously in the Barcode ligation and Multiplexing section.

### Data Analysis

Libraries were sequenced on the Illumina Hi-Seq or NEXT-seq platform. First, paired end reads were split by barcode sequence using Je demultiplex with options BPOS=BOTH BM=READ_1LEN=4:6 FORCE=true C=false 6. BM and LEN options were adjusted for samples with a 3 nt barcode instead of 4, and for samples where the barcode is located in read 2. Barcode sequences are available on Github at https://github.com/ckatanski/Q_paper. Next, read 2 files were used to map with bowtie2 (45) with the following parameters:-q-p 10--local—no-unal. Reads were mapped to curated list of non-redundant tRNA genes with tRNAScane score >40 for respective organisms (human and E. coli). Bowtie2 output sam files were converted to bam files, then sorted using samtools. Next, IGV was used to collapse reads into 1 nt window. IGV output.wig files were reformatted using custom python scripts (available on GitHub at https://github.com/ckatanski/Q_paper). The bowtie2 output Sam files were also used as input for a custom python script using PySam, a python wrapper for SAMTools (46) to sum all reads that mapped to each gene. Data were visualized with custom R scripts (available on GitHub at https://github.com/ckatanski/Q paper). “Reads per million” normalization was calculated by dividing the number or reads mapped to a specific gene by the total number of tRNA-mapped reads in that sample, and scaling by a factor of 1,000,000. Unless otherwise stated, analysis was limited to genes and positions with read coverage >100 reads. For presentation, the position value of each tRNA gene was adjusted to match canonical tRNA numbering (anticodon in positions 34, 35, 36). For calibration curve, Origin was used to fit linear or semilogy line of best fit using least squares regression and calculated r2 statistics. For comparing change in the modifications during stress, an unpaired two-sided Wilcox test (Mann-Whitney) was used, in which p-values were: ns, >0.05; *, less than 0.05; **, less than 0.01, and ***, less than 0.001.

### Results

### Periodate Treatment Produces Deletion Signatures for Q-Modification in RNA Sequences

Using total RNA from HEK293T cells that are either completely devoid of tRNA Q-modification (0Q), or fully modified with Q (100Q) (FIG. 1B), it was unexpectedly found that tRNA^{His} from 100Q cells showed a deletion signature at the Q34 nucleotide only in the periodate-treated, but not in the untreated, control sample (FIGS. 1C, D). The signature is absent in 0Q cells (FIGS. 1C, D). The periodate-dependent deletion signature of the Q nucleotide in sequencing is the most pronounced among the other signatures analyzed such as mutations, insertions, and stops (FIG. 1E).

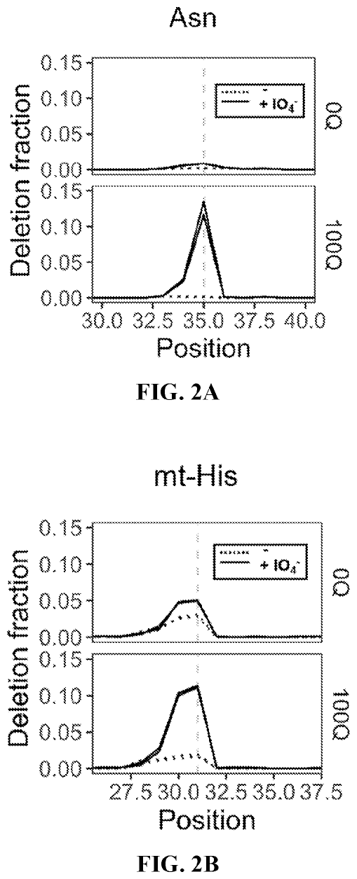

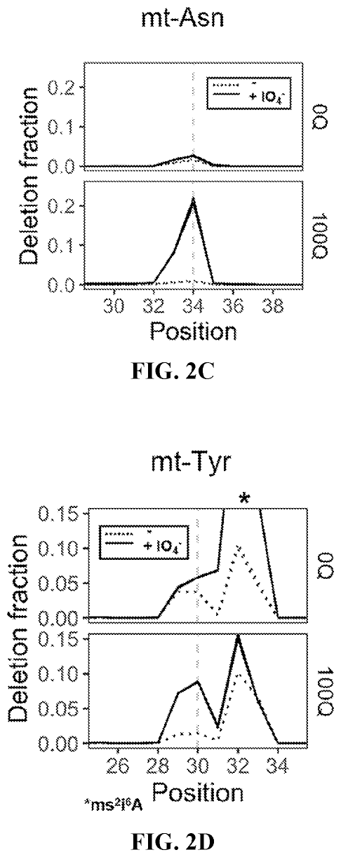

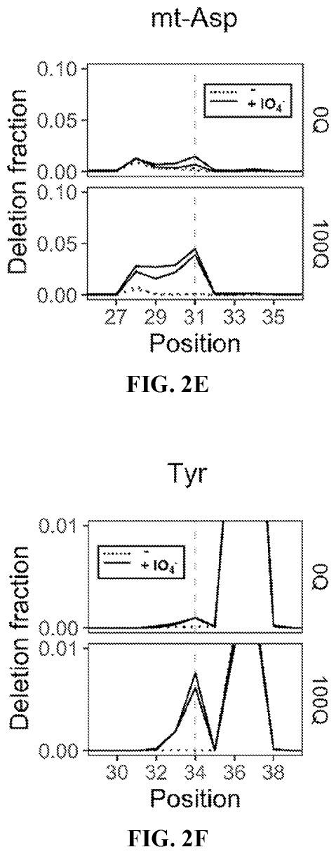

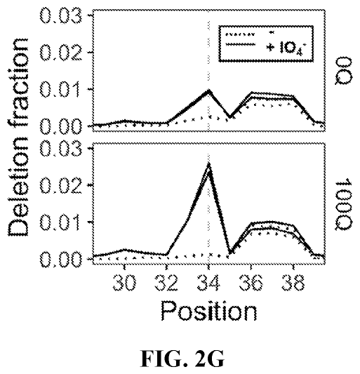

FIG. 2 shows the use of periodate treatment to produce sequencing signatures in DNA copies of additional base-modified RNAs.

A total of 8 tRNAs in human cells can be modified with Q. The nuclear-encoded tRNA^{His}and tRNA^{Asn}are modified with Q, whereas tRNA^{Tyr}and tRNA^{Asp}are further modified by glycosylation to galactosyl-Q and mannosyl-Q, respectively. The mitochondrial-encoded tRNAs for these same 4 amino acids are also modified with Q. First, the presence of deletion signatures in DNA sequences copied from Q-modified tRNAs were examined. The cytosolic tRNA^{Asn}displayed a relatively high deletion fraction of ˜ 13% at the Q34 position that is Q-modification-and periodate treatment-dependent (FIG. 2A). All 4 mitochondrial tRNAs show deletion signatures in the same manner at the Q-modified nucleotide as well, ranging from ˜4% detection level in mt-tRNA^{Asp}to ˜20% detection level in mt-tRNA^{Asn}(FIGS. 2B-D).

Next, the ability of known ms²i⁶ A mutations at position 37 in tRNAs to produce deletion signatures in DNA sequencing preceded by periodate treatment was examined. Deletion signatures were found associated with ms²i⁶ A in mitochondrial-encoded tRNA^{Asp} (FIG. 2E), and with nuclear-encoded tRNA^{Tyr} (FIG. 2F). Another known modification that also produces deletion signature is m1G at position 37, shown in FIG. 2G for nuclear-encoded tRNA^{Asp}. The periodate-independent deletion signature at position 37 seen in FIG. 2G, 0Q is of unknown origin.

These results demonstrate that deletion signatures are associated with Q34, ms²i⁶ A37 and m1G modification in tRNAs.

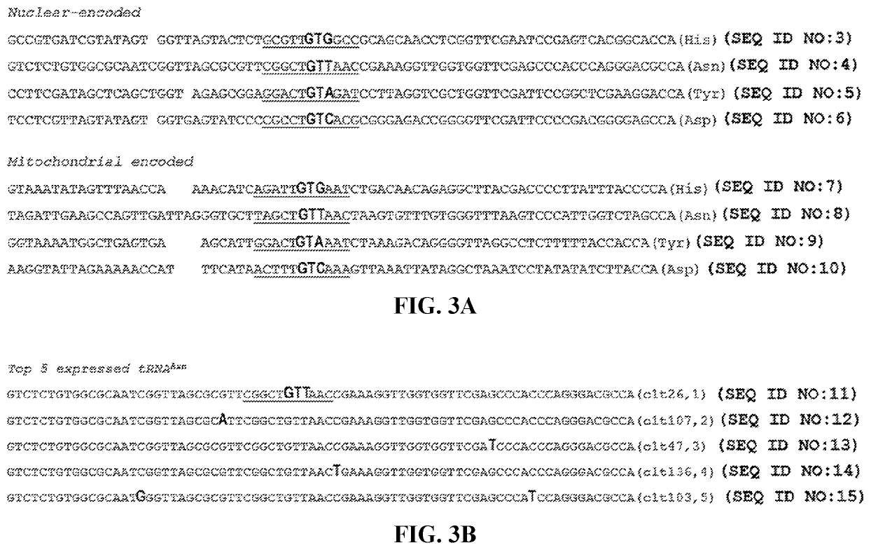

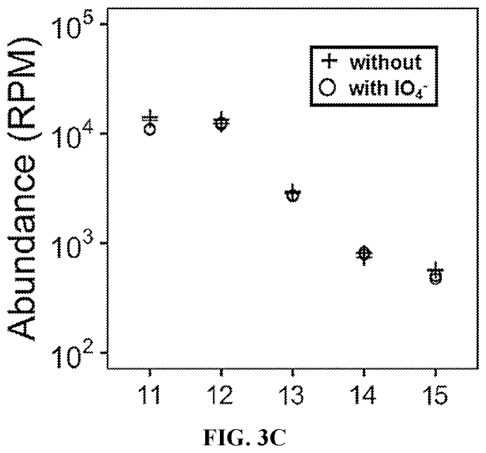

FIG. 3 shows the effect of the base sequences surrounding the Q-modified nucleotide on the deletion signature detected.

A factor that may affect the level of deletion signature is the nucleotide sequence immediately upstream of the Q34 residue. It was found that cytosolic tRNA^{Asn}, which has an upstream C32 (5′GGCUQUU) (FIG. 3A), has a high deletion fraction (FIG. 2A), whereas tRNA^{His} with an upstream U32 (5′CGUUQUG) (FIG. 3A) has a low deletion fraction (FIG. 1D). Similarly, mt-tRNA^{Asn} (5′AGCUQUU) (FIG. 3A) has upstream C32 and mt-tRNA^{His} (5′GAUUQUG) (FIG. 3B) has upstream U32, which is consistent with the observed high and low deletion fraction for these two tRNAs (FIGS. 2C and 2B, respectively). mt-tRNA^{Asp} (5′CUUUQUC) has upstream U32 and U31 (FIG. 3A), which may correlate with the observed deletion signature spanning 4 nucleotides (FIG. 2E). Finally, mt-tRNA^{Tyr} (5′GACUQUA) could have a high deletion fraction, but this may be obscured by the effects of ms²i⁶ A37 modification, which results in a large deletion signature that overwhelms the Q-modification deletion signature, and is detected in samples both with and without periodate treatment (FIG. 2D).

These results demonstrate that the deletion fraction detected in the DNA sequence copied from a Q-modified tRNA is strongly dependent on the upstream sequence context of the Q-modification.

This experiment explores whether periodate treatment results in deletion signatures in DNA sequences copied from glycol-Q-modified tRNAs.

The glyco-Q-modified tRNAs do not significantly react with the boronic acid derivative used in APB gels, and so do not demonstrate a shift in gel migration distance like that seen for Q-modified tRNAs (see, e.g., FIG. 1B). However, both galactose and mannose can form a small proportion of furanose tautomer containing a cis-diol in equilibrium with the major pyranose tautomer. The 100Q samples for tRNA^{Tyr} and tRNA^{Asp} are known to have nearly stoichiometric amount of glycosylated Q-modification, as measured by a combination of APB and acid denaturing gel electrophoresis (not shown). It was found that both nuclear-encoded tRNA^{Tyr} and tRNA^{Asp} display deletion signatures using periodate treatment in the DNA sequencing system (FIGS. 2F-2G), although the deletion fractions detected were small. The deletion fractions were only ˜0.5% and 2% for tRNA^{Tyr} and tRNA^{Asp}, respectively, which are substantially lower than the values seen for unglycosylated Q-modified tRNAs. Since the deletion background in the sequencing is <0.1%, these low deletion fractions are still useful in detecting glyco-Q modifications, especially in tRNA sequencing where the read coverage at the glyco-Q nucleotides can easily reach >1,000. While the differences in the deletion fraction for cytosolic tRNA^{Tyr} and tRNA^{Asp} may be related to the periodate-reacted product of gal-Q and man-Q, both tRNAs also have C32 in their upstream sequences (tRNA^{Tyr} has 5′GACUGUA, tRNA^{Asp} has 5′GCCUGUC), which enhances the fraction of deletion signatures detected.

Together, these results confirm that modified nucleotides, in particular Q-modified nucleotides, can readily be detected using periodate-treated RNA-seq libraries, with glyco-Q modified nucleotides having a lower detection signature.

### Deletion Fraction can be Used to Quantify Q-Modification Levels

These experiments explore using cDNA sequencing to quantify Q-modification fraction in a biological sample, which would enable simultaneous assessment of transcriptome-wide tRNA properties associated with Q-modification.

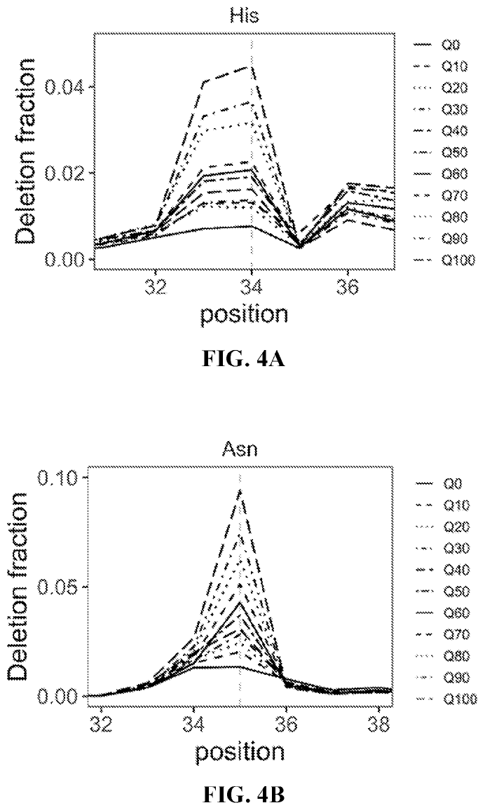

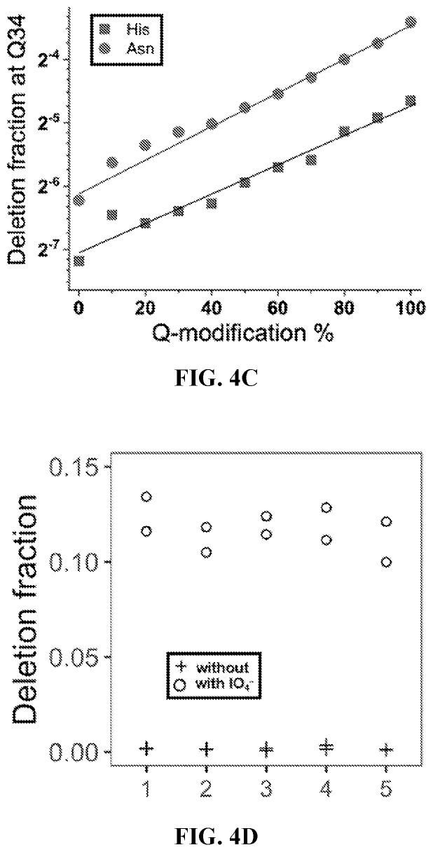

To assess whether the deletion signature can be used to quantify Q-levels, two biological samples of 0Q and 100Q HEK293T cells were systematically mixed to provide varying ratios between 0% and 100% Q, and sequencing reactions were performed after periodate treatment of total RNA. The deletion fraction at the Q34 position detected for tRNA^{His} and tRNA^{Asn}steadily increased as the cell mixture had an increasing proportion of 100Q RNA (FIGS. 4A,B). Calibration curves were prepared from the data (FIG. 4C).

It was found that curve fitting of the changes in the deletion fraction versus the % Q modification is much better when an exponential dependence of the Q-modification fraction is used (r2=0.964, 0.985, FIG. 4C) rather than a linear fit (r2=0.901, 0.923, not shown). An explanation of this non-linear result is that (Q34) tRNA is reverse-transcribed less efficiently after periodate treatment than unmodified (G34) tRNA is. In any given sample, at most 20% of Q34-modified tRNAs produce a deletion signature. Therefore, the quantitative production of DNA copies containing deletion signature is skewed when the level of Q-modification in a sample is low. Support for this explanation is provided by similar non-linear results having been observed for other modifications that reduce the efficiency of reverse transcriptase, such as for N1-methyl-A (m1A) RNA.

Interestingly, both tRNA^{His} and tRNA^{Asn} show a very similar slope in their Q-modification calibration curves (FIG. 4C). This result is consistent with the absolute value of the detected deletion fraction being dependent on the sequence context surrounding the Q34 within each type of tRNA, but with the changing Q-levels of the population of tRNAs in each sample responding in the same way to the reverse transcriptase reaction.

Five tRNA^{Asn} isodecoders comprise >95% of total tRNA^{Asn} (FIGS. 3C, D), whereas a single tRNA^{His} isodecoder comprises >99% of total RNAHis in the HEK293T RNA samples. The deletion fractions detected for 100Q samples of the 5 tRNA^{Asn} isodecoders are nearly identical (FIG. 4D), indicating all are modified at the same level. This is consistent with all 5 isodecoders sharing the identical sequence in the 11 nucleotide window around the Q34 residue (FIG. 3B, region underlined for tRNA clt26, 1).

These results show that the levels of deletion fraction detected in periodate-treated RNA-seq libraries can be used to quantify the Q-modification levels in the RNA samples.

### Periodate Treatment Also Produces Sequencing Signatures in 2-Thio-Modifications

These experiments explore whether the presence of 2-thio modifications in RNAs produce sequencing signatures in the DNA copies made from them.

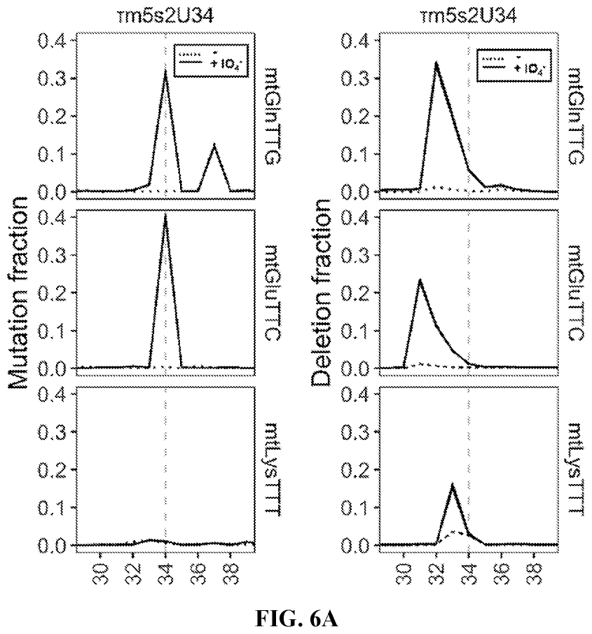

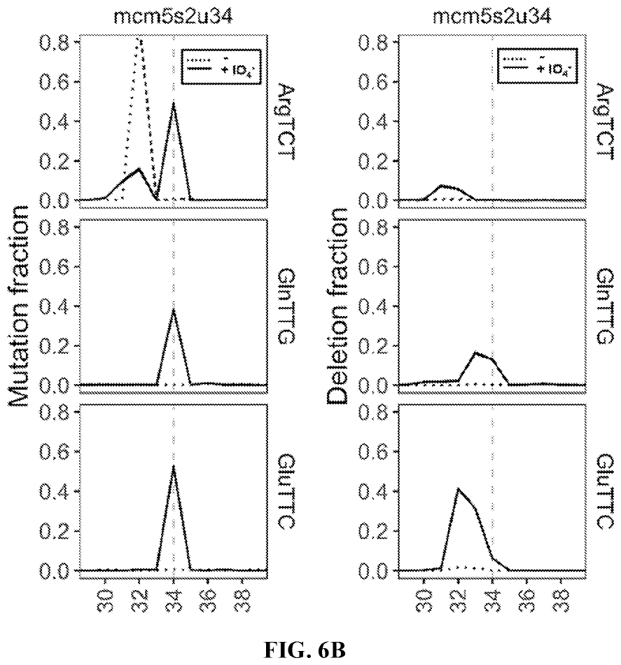

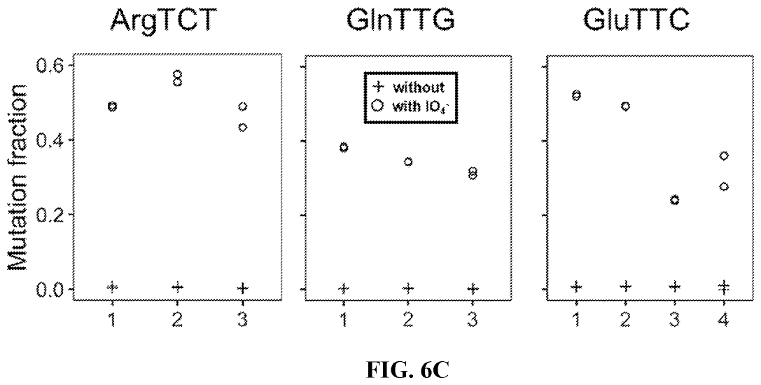

In mitochondrial-encoded tRNAs, the s²U-modification is present in the wobble anticodon position of tRNAGIn, tRNAGlu, and tRNALys, in the context of 5-taurinomethyl-2-thiouridine (τm⁵s²U). A strong DNA mutation signature was found for mt-tRNA^{Gln} and mt-tRNA^{Glu} at the modified nucleotide (FIGS. 5B and 6A, left), accompanied by a strong double deletion signature 1-2 nucleotides upstream from the modified nucleotide (FIGS. 5B and 6A, right). Both modification signatures are periodate-dependent but not Q-dependent (dotted and solid lines represent the absence and presence of periodate, respectively). Mitochondrial-tRNA^{Lys} shows a periodate-dependent deletion signature consistent with a 2-thio modification (FIG. 5B and 6A, right), but no mutation signature (FIG. 5B and 6A, left). This result may be due to unusual sequence context and/or other modifications around the 5-methyltaurine modified nucleotide. Among the τm⁵s²U34 modified tRNAs, only mt-tRNA^{Lys} has a N6-threonylcarbamoyladenosine (t6A) modification at position 37, which may influence whether an s² mutation signature at wobble position 34 is obtained in the reverse transcriptase reaction. Another possibility is that the mt-tRNA^{Lys} in the specific sample (total RNA from HEK293T cells) may not contain a 2-thio modification at the U34position.

Importantly, the two mitochondrial tRNAs that have 5-methyltaurine, but no 2-thio modification, i.e., mt-tRNA^{Leu} (TAA) and mt-tRNA^{TIP}, do not show periodate-dependent mutation or deletion signatures at the τm⁵U wobble position 34 (FIG. 5C), which lends support to the conclusion that the periodate-dependent mutation and deletion in mt-tRNA^{Gln} nd mt-tRNA^{Glu} are indeed derived from the 2-thio modification. Unexpectedly, strong periodate-dependent mutation and deletion signatures are present in mt-tRNA^{TIP} (FIG. 5C). These signatures apparently correspond to the known 2-methylthio-N6-isopenteny-A (ms²i⁶A)37 modification in this tRNA. The sulfur atom in ms²i⁶ A may also be subject to the thio-modification, which likely contributes to the periodate-dependent signatures in sequencing.

In nuclear-encoded tRNAs, the s2-modification is present in the wobble anticodon position of tRNA^{Arg} (TCT), tRNA^{Gln} (TTG), and tRNA^{Glu} (TTC) in the context of 5-(carboxy) methylaminomethyl-2-thiouridine (mnm⁵s²U34). Indeed, a strong periodate-dependent mutation signature at the location of the modified nucleotide was found for all three tRNAs in both 0Q and 100Q cells (FIGS. 5D and 6B, respectively), indicating that the signature is not Q-dependent. A double deletion signature is also present on or upstream of the modified nucleotide depending on the tRNA species. These results indicate that periodate treatment is capable of detecting mnm⁵s²U34 modifications, although the fraction of mutation and deletion signatures seen likely depends on the context of the neighboring sequences and other modifications close to the 2-thio modifications.

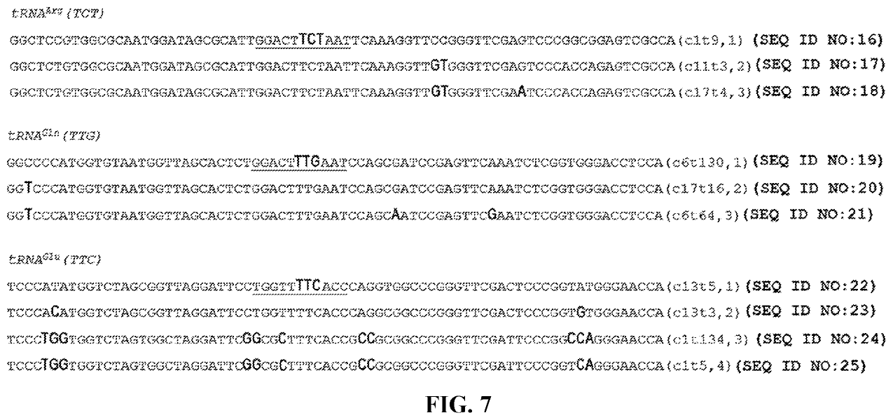

The mutation signatures for the abundant tRNA^{Arg} (TCT), tRNA^{Gln} (TTG), and tRNA^{Glu} (TTC) isodecoders in the samples were also compared for both 0Q and 100Q RNAs (FIGS. 5E and 6C, respectively). Among the isodecoders of tRNA^{Arg} (TCT) and tRNA^{Gln} (TTG), the mutation fractions are comparable to each other despite variations in abundance of the isodecoders. This result could be predicted, as the sequence differences of these isodecoders are all outside of the window ±5 nucleotides of the modification (FIG. 7). In contrast, an ˜2.5-fold difference in mutation fraction was observed among the tRNA^{Glu} (TTC) isodecoders (FIGS. 5E and 6C). This difference may be attributed to the substantial differences seen among the sequences of these tRNA^{Glu} (TTC) isodecoders within the ±5 nucleotide window of the modification (FIG. 7), rather than to a difference of the mnm⁵s²U34 modification fraction.

Calibration curves for 2-thio-modifications can also be prepared. They can be readily obtained upon chemical synthesis of oligonucleotides containing these modifications.

These results show that 2-thio-modifications in nucleotides produce both mutation and deletion signatures in DNA copies of the RNA sequences.

Thio-Modifications in E. coli and in Stress Response

These experiments explore the sequencing effects of thio modifications in the tRNAs of E. coli.

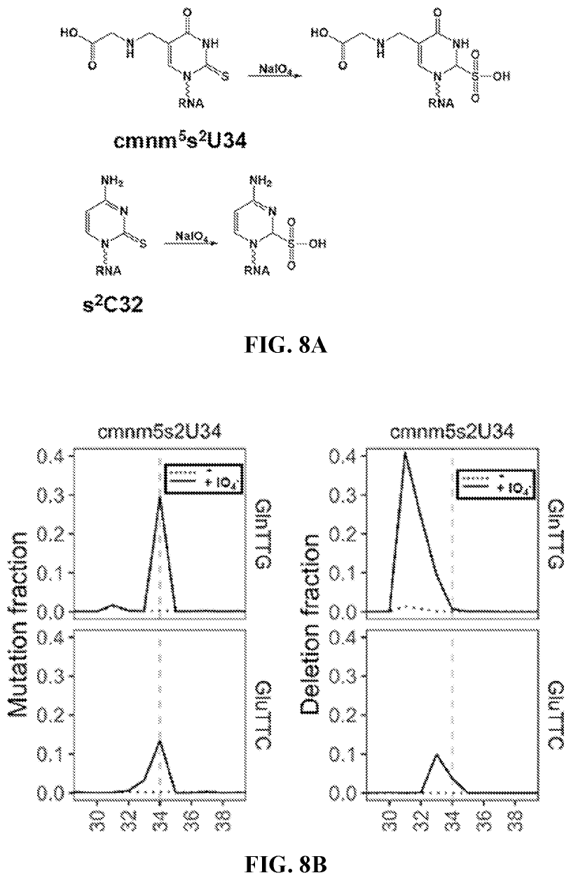

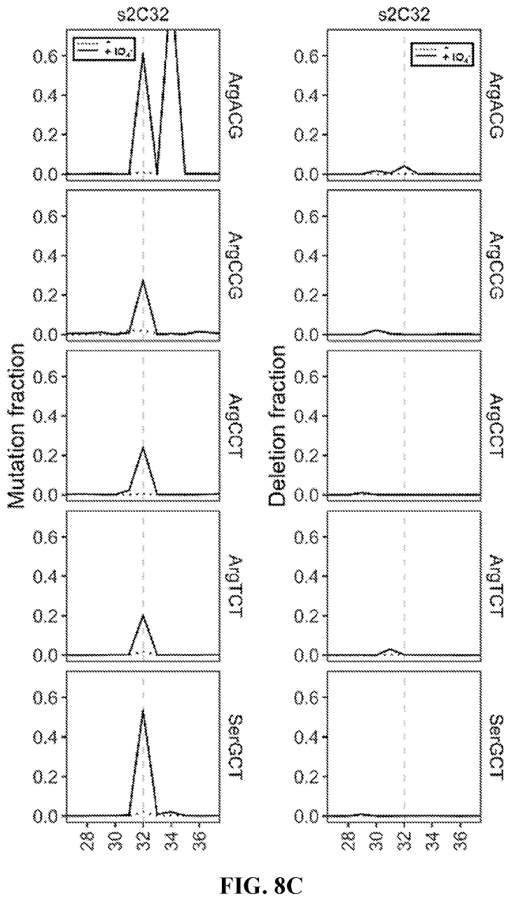

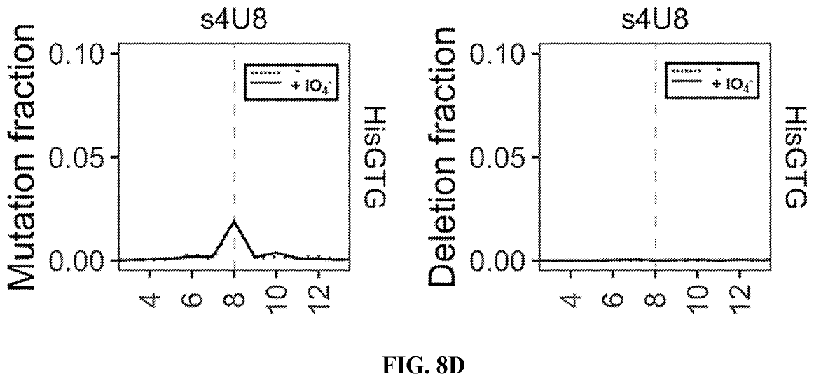

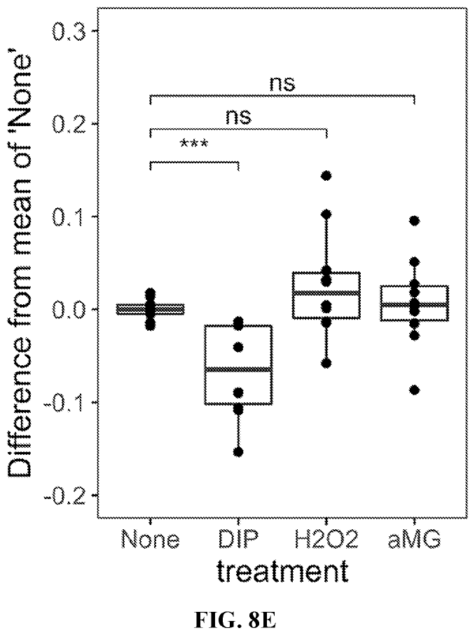

2-thio modifications of the types shown in FIG. 8A are present in E. coli tRNAs. Sequencing of E. coli tRNA was performed, with and without periodate treatment. The sequences displayed strong, periodate-dependent mutation and deletion signatures for the known 5-carboxymethylaminomethyl-2-thiouridine (cmnm⁵s²U) modification at the wobble anticodon position in tRNA^{Gln} (TTG) and tRNA^{Glu} (TTC) (FIG. 8B). As found for several of the human 2-thio modifications, the mutation signature is located at the modified nucleotide, whereas the deletion signature is at or immediately upstream of the modification nucleotide.

E. coli tRNA also contains 2-thio-C (s²C) and 4-thio-U (s⁴U) modifications that are absent in human tRNA. Strong, periodate-dependent mutation signatures were found for the known s²C32 modification in all 5 tRNAs, at the location of the modified nucleotide (FIG. 8C, left panels). In each case, a low level of deletion signature was also observed around 1-3 nucleotides upstream of the s²C modified nucleotide (FIG. 8C, right panels). On the other hand, s⁴U modification at position 8 shows only a mutation signature that is independent of periodate treatment, and no deletion signature (FIG. 8D). A possible explanation is that the structure of the 2-thio modification is located in the minor groove of the DNA-RNA hybrid in the active site of reverse transcriptase, whereas the 4-thio modification structure is located in the major groove. These results are consistent with a thio-oxidation structural group located in the minor groove of the RNA-DNA hybrid interfering with the proof-reading activity of reverse transcriptase.

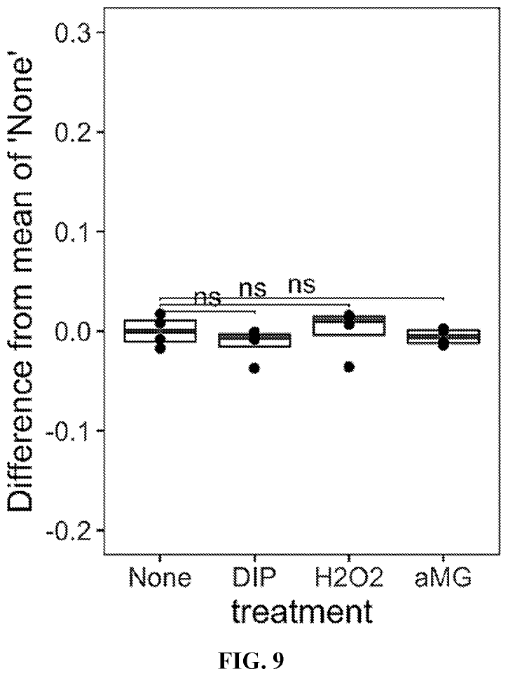

To examine biological consequences of 2-thio-modifications in tRNAs, E. coli were grown in the presence of various stressors: 2,2′-dipyridyl (DIP), which chelates Fe²⁺; H₂O₂, which induces oxidative stress; and α-methyl glucoside-6-phosphate (αMG), which induces glucose starvation. tRNA sequencing was performed on both control (unstressed) and stressed samples. The mutation rates for the cmnm⁵s²U34-modified tRNA^{Gln}(TTG) and tRNA^{Glu}(TTC) did not change under the stress conditions (FIG. 9). In contrast, the s²C32 levels in all tRNAs were reduced in response to DIP stress, but not in response to H₂O₂ or αMG stress (FIG. 8E). The 2-thio-C modification is produced by the enzyme TtcA, which contains an iron-sulfur cluster in the active site; a lower production of s²C32 under conditions of iron chelation is consistent with a reduction in activity of the TtcA enzyme. It remains to be determined whether the reduction of s²C32 level in certain tRNAs affects the decoding of specific codons (CGN and AGN) read by these modified tRNAs.

These results demonstrate that 2-thio modifications in bacterial (E. coli) RNA produce the same deletion and mutation signatures in DNA copies of the RNA sequences as found for human RNAs, and provide another way to explore the consequences of stresses experienced by the E. coli.

### EXAMPLE 2

### Methods

The following methods were used in analysis of base modifications in small RNAs of a human stool sample, in accordance with aspects of the disclosure.

### Read Processing and Mapping

Libraries were sequenced on Illumina Hi-Seq or NEXT-seq platform. First, paired end reads were split by barcode sequence using Je demultiplex with options BPOS=BOTH BM=READ_1 LEN=4:6 FORCE=true C=false (Girardot, C., Scholtalbers, J., Sauer, S., Su, S. Y. and Furlong, E. E. (2016) Je, a versatile suite to handle multiplexed NGS libraries with unique molecular identifiers. BMC Bioinformatics, 17, 419). BM and LEN options were adjusted for samples with a 3 nt barcode instead of 4, and for samples where the barcode is located in read 2.Next, only the read beginning with the barcode (usually read 2) was used to map with bowtie2(version 2.3.3.1) with the following parameters: “-q-p 10--local--no-unal”. Human sample reads were mapped to the human transcriptome, with tRNA genes shaped for a curated, non-redundant, set of high-scoring tRNA genes. This reference was a combination of HG19 ORFs, ncRNAs, and a curated tRNA list based on HG19 tRNAs curated to be non-redundant, tRNA-scan SE with score >47, and 3′ “CCA” appended. Bowtie2 output sam files were converted to bam files, then sorted using samtools. Next IGV was used to collapse reads into 1 nt window. IGV output.wig files were reformatted using custom python scripts (available on GitHub). The bowtie2 output Sam files were also used as input for a custom python script using PySam, a python wrapper for SAMTools (see Li, H., Handsaker, B., Wysoker, A., Fennell, T., Ruan, J., Homer, N., Marth, G., Abecasis, G., Durbin, R. and Genome Project Data Processing, S. (2009) The Sequence Alignment/Map format and SAMtools. Bioinformatics, 25, 2078-2079), https://github. com/pysam-developers/pysam) to sum all reads that mapped to each gene. Related custom scripts were used to divide reads based on which 10 nt window the 3′ end mapped to for each tRNA; this is for fragment analysis. Data was visualized with custom R scripts. All custom scripts are available on GitHub (https://github. com/ckatanski/CHRIS-seq).

Microbiome 5S rRNA Analysis

Reference sequences for 5S rRNA were downloaded from the 5S rRNA database (http://combio. pl/rrna/) (Szymanski, M., Zielezinski, A., Barciszewski, J., Erdmann, V. A. and Karlowski, W. M. (2016) 5SRNAdb: an information resource for 5S ribosomal RNAs. Nucleic Acids Res., 44, D180-183). Sequences were combined from Bacteria (n=7291), Archaea (n=319), Eukaryota (n=2861), mitochondria (n=110), and plastids (n=838). Full lineages were assigned to each reference using the ETE3 NCBITaxa toolkit in python. Sequencing reads were processed and mapped to the combined 5S reference set using bowtie2 with the mapping parameters as described above. Mapping data were further processed for base-wise mapping information as well as by-gene counting. These data were combined with SRP signal recognition particle mapping data (below) by species. Mapping data for species within the same genus was summed. Data was then grouped by order or class, respectively, then summed.

Microbiome tRNA Modification Analysis

Sequencing reads were aligned to the tRNA references from the 7 microbes with most abundant tRNA coverages. The tRNA references were obtained from Genomic tRNA database (4) from these species: http://gtrnadb. ucsc. edu/GtRNAdb2/genomes/bacteria/Bact_dore_1/;http://gtrnadb. ucsc. edu/GtRNAdb2/genomes/bacteria/Bifi_long_longum_BBMN68/:http://gtrnadb. ucsc. edu/GtRNAdb2/genomes/bacteria/Carn_malt_LMA28/: http://gtrnadb. ucsc. edu/GtRNAdb2/genomes/bacteria/Clos_beij_ATCC_ 35702_SA_1/Clos_beij_ATCC 35702_SA_1-seq.html: http://gtrnadb. ucsc. edu/GtRNAdb2/genomes/bacteria/Faec_prau_L2_6_L2_6/;http://gtrnadb.ucsc. edu/GtRNAdb2/genomes/bacteria/Lach_phyt ISDg/; http://gtrnadb. ucsc.edu/GtRNAdb2/genomes/bacteria/Rose_inte_XB6B4/Rose inte_XB6B4-seq.html. (See Chan, P.P. and Lowe, T.M. (2016) GtRNAdb 2.0: an expanded database of transfer RNA genes identified in complete and draft genomes. Nucleic Acids Res, 44, D184-189.) Modifications were identified by either deletion or mutation signatures according to Katanski, C. D., Watkins, C. P., Zhang, W., Reyer, M., Miller, S. and Pan, T. (2022) Analysis of queuosine and 2-thio tRNA modifications by high throughput sequencing. Nucleic Acids Res., 50, e99.

### Results

Abundance of Microbial 5S rRNA Sequences

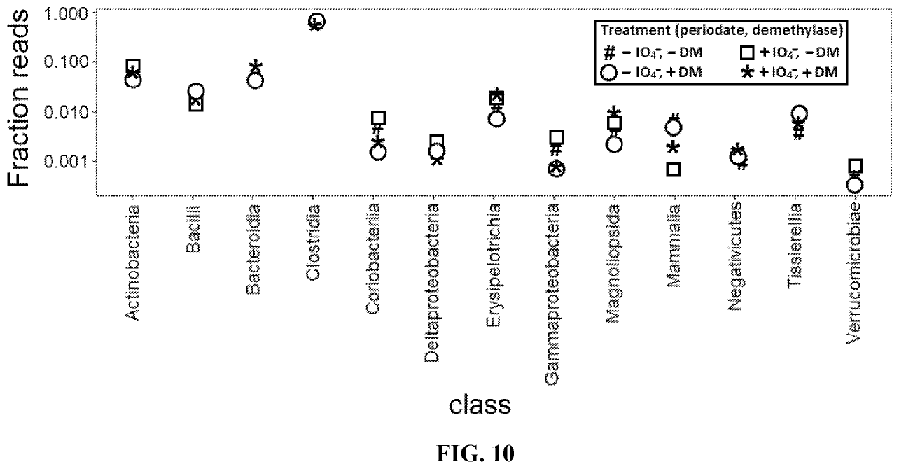

FIG. 10 shows the abundance of microbial 5S rRNAs from different bacterial taxa at the class level. Libraries were constructed for the same human stool sample under four treatment conditions: # is minus periodate, minus demethylase; square is plus periodate, minus demethylase; circle is minus periodate, plus demethylase; and * is plus periodate, plus demethylase.

5S rRNA abundance was the same in all 4 sequencing libraries, indicating that periodate and demethylase treatments did not affect 5S rRNA abundance.

IRNA Q-Modifications Using Reference Sequences from Various Bacterial Species

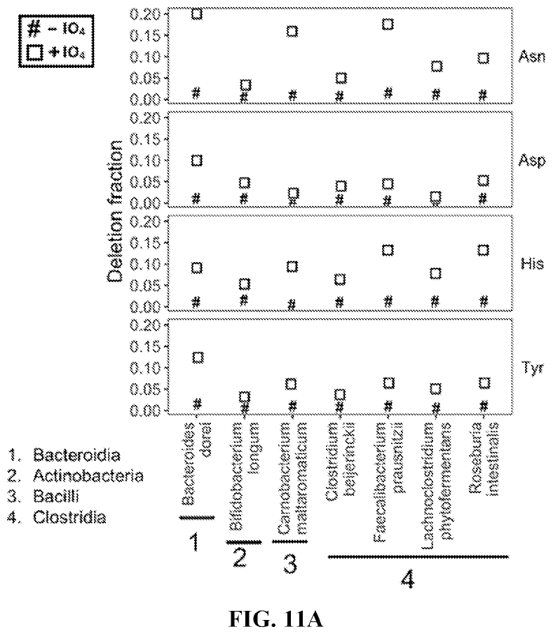

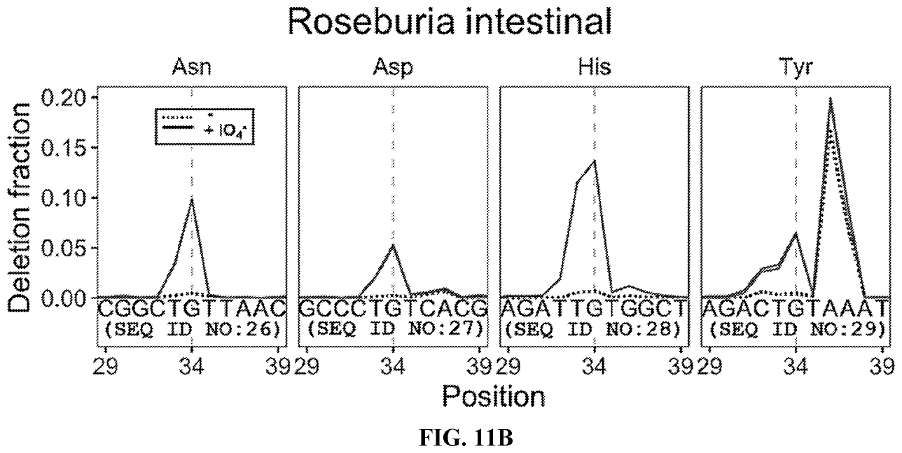

FIG. 11 shows tRNA Q-modifications (only present in tRNAs for Tyr/His/Asn/Asp) found in reference sequences from the bacterial species indicated. Sequence reads were performed with and without periodate the treatment. Deletions at Q-modifications became became detectable upon periodate treatment. FIG. 11A shows the deletion fraction at nucleotide position 34 (tRNA nomenclature, wobble anticodon) for tRNAs from species within 4 bacterial classes: bacteroidia, actinobacteria, bacilli, clostridia. Sequence reads were performed under the treatments: # is minus periodate, square is plus periodate. FIG. 11B shows the deletion fractions within a region +/−5 nucleotide of position 34, in the Q-modifiable tRNAs in the bacterial genus Roseburia: dotted line—minus periodate; solid line—plus periodate.

Deletion fractions detected at tRNA Q-modifications varied among the tRNAs and bacteria tested. The large deletion at position A37 in tRNA^{Tyr}(FIG. 11B) is from a putative ms²i⁶A modification.

IRNA s²U Modifications Using Reference Sequences from Various Bacterial Species

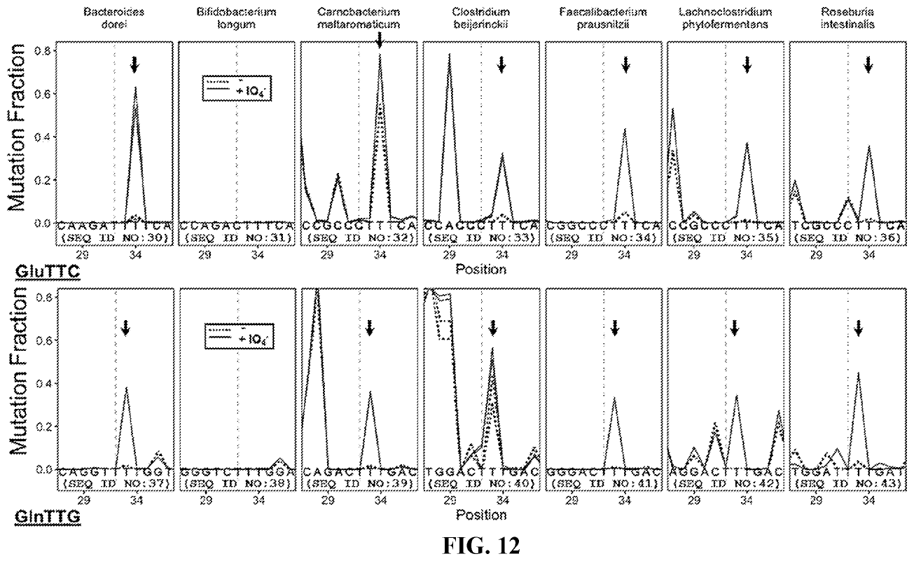

FIG. 12 shows tRNA s²U modifications found in reference sequences from the bacterial species indicated. Mutation fractions detected in tRNA^{Glu}(TTC) (top) or tRNA^{Gln}(TTG) (bottom) sequences around position U34 (tRNA nomenclature) are shown. S²U was detected by an increased mutation rate at the U34 position upon periodate treatment, indicated by bold arrows. Dotted line is minus periodate; solid line is plus periodate.

Some level of S²U modification was detected in both Glu (TTC) (top) and Gln (TTG) tRNAs from every bacterial species tested except for B. longum, in which neither tRNA exhibited S²U modification.

RNA s²C32 Modifications Using Reference Sequences from Various Bacterial Species

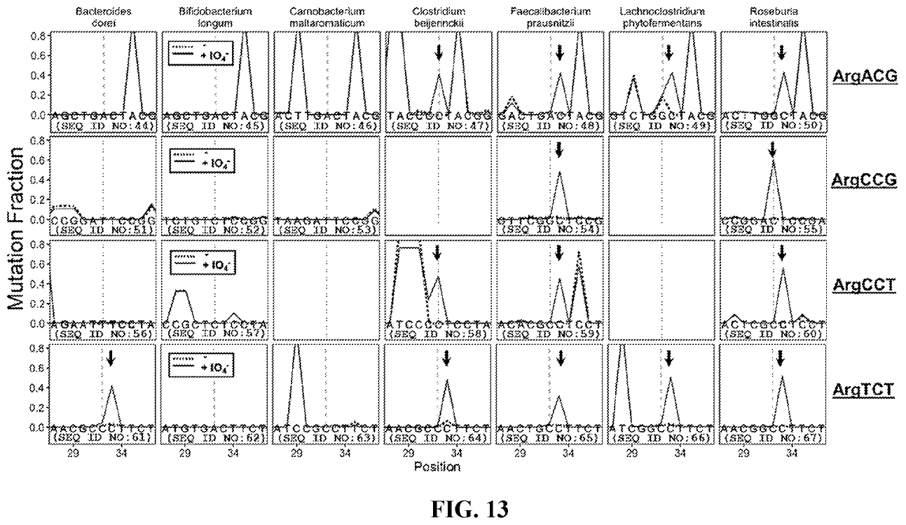

FIG. 13 shows tRNA s²C32 modifications found in reference sequences from the bacterial species indicated. For Bacteroides dorei Arg (CCT), the sequence listed in FIG. 13 is one of two tRNA isodecoder sequences. The other sequence is AAGTTTCCTAA (SEQ ID NO: 73). In the specific microbes studied, some tRNAs of the corresponding anticodons are absent so there are no sequences for them. For example, there is no tRNA^{Arg}(CCG) in C. beijerinckii, or L. phylofermentans. Mutation fractions detected in tRNA^{Arg}(ACG), tRNA^{Arg}(CCG), tRNA^{Arg} (CCT), and tRNA^{Arg}(TCT) sequences around position C32 (tRNA nomenclature) are shown. S²C was detected by an increased mutation rate at the C32 position upon periodate treatment, indicated by arrows. Dotted line is minus periodate; solid line is plus periodate.

In addition to the four tRNA^{Arg}s shown in FIG. 13, s²C32 modification was also identified in other tRNAs in the sample (data not shown): B. dorei, GlyTCC, TrpCCA; C. maltaromaticum, CysGCA, SerGCT, SerTGA; C. beijerinckii, CysGCA, GlyTCC, SerGCT, SerTGA; F. prausnitzii, GlyTCC, SerGCT, TyrGTA, L. phytofermentans, GlyTCC, TyrGTA; and R. intestinalis, CysGCA, GlyTCC, SerGCT, TrpCCA, TyrGTA.

The fraction of s²C32 modifications varied from none detected in any of the 4 tRNA^{Arg}s tested in a species (e.g., in C. maltaromanticum) to the modification detected in all 4 tRNA^{Arg}s tested (in F. prausnitzi and R. intestinalis).

IRNA s²C34 Modifications Using Reference Sequences from Various Bacterial Species

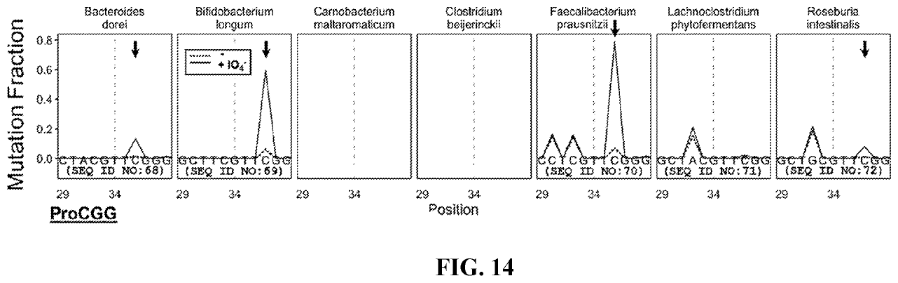

FIG. 14 shows tRNA s²C34 modifications found in reference sequences from the bacterial species indicated. In the specific microbes studied, some tRNAs of the corresponding anticodons are absent, so there are no sequences for them. For example, there is no tRNA^{Pro}(CGG) in C. maltaromaticum or C. beijerinckii. Mutation rates detected in tRNA^{Pro}(CGG) sequences around position C34 (tRNA nomenclature) are shown. S²C was detected by an increased mutation rate at the C34 position upon periodate treatment, indicated by arrows. Dotted line is minus periodate; solid line is plus periodate.

In addition to the tRNA^{Pro}(CGG) data shown in FIG. 14, s²C34 modifications were also identified in these tRNAs in the sample (data not shown): B. dorei, ThrCGT; B. longum, AlaCGC, LysCTT, GlnCTG, ThrCGT, ValCAC; C. beijerinckii, GlnCTG; and F. prausnitzii, AlaCGC, ThrCGT.

S²C modifications were detected in the tRNA^{Pro}(CGG) from 4 of the 8 species tested.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting a nucleotide modification in a sample comprising RNA, the method comprising: reacting RNA in the sample with periodate to form periodate-treated RNA in the sample, sequencing periodate-treated RNA in the sample, and detecting a modification signature in the sequence, wherein the nucleotide modification is (a) substitution with queuosine (Q-modification) or (b) substitution of an oxygen atom at the 2-position of a pyrimidine nucleotide with a sulfur atom (2-thio modification).

2. The method of claim 1, further comprising performing a control sequencing reaction on a portion of RNA from the sample, wherein the control sequencing reaction is performed on RNA not treated with periodate.

3. The method of claim 1, wherein the modification signature detected is the presence of a mutation signature or a deletion signature in the sequence.

4. The method of claim 1, wherein the nucleotide modification is Q-modification.

5. The method of claim 4, wherein the presence of a deletion signature in the sequence at the site of Q-modification is detected.

6. The method of claim 4, further comprising quantifying the fraction of RNA having Q-modification.

7. The method of claim 6, wherein quantifying the fraction of RNA comprises comparing a detected Q-level in the RNA to a calibration curve established from RNAs with no Q-modification (0Q) and with full Q-modification (100Q).

8. The method of claim 1, wherein the nucleotide modification is 2-thio modification.

9. The method of claim 8, wherein the presence of (i) a mutation signature in the sequence at the site of the 2-thio modification and/or (ii) a deletion signature in the sequence near the site of the 2-thio modification is detected.

10. The method of claim 1, wherein the RNA is from a mammal or a bacterium.

11. The method of claim 1, wherein the RNA is total RNA, tRNA, nuclear-encoded tRNA, or mitochondrial-encoded tRNA.

12. The method of claim 1, wherein the periodate-treated RNA is sequenced using high throughput DNA sequencing.

13. The method of claim 2, wherein the nucleotide modification is Q-modification.

14. The method of claim 13, wherein the presence of a deletion signature in the sequence at the site of Q-modification is detected.

15. The method of claim 13, further comprising quantifying the fraction of RNA having Q-modification.

16. The method of claim 15, wherein quantifying the fraction of RNA comprises comparing a detected Q-level in the RNA to a calibration curve established from RNA with no Q-modification (0Q) and with full Q-modification (100Q).

17. The method of claim 2, wherein the nucleotide modification is 2-thio modification.

18. The method of claim 17, wherein the presence of (i) a mutation signature in the sequence at the site of the 2-thio modification and/or (ii) a deletion signature in the sequence near the site of the 2-thio modification is detected.

19. The method of claim 2, wherein the RNA is from a mammal or a bacterium.

20. The method of claim 2, wherein the RNA is total RNA, tRNA, nuclear-encoded tRNA, or mitochondrial-encoded tRNA.

21. The method of claim 2, wherein the periodate-treated RNA is sequenced using high throughput DNA sequencing.
